(19) United States
(12) Patent Application Publication
(10) Pub. No.: US 2026/0224190 A1
(43) Pub. Date: Aug. 6, 2026

(54) Systems and Methods For Jugular Vein Measurement Using Ultrasound

(71) Applicant: Foundry Innovation & Research 1, Ltd., Dublin (IE)

(72) Inventors: Fiachra M. Sweeney, Dublin (IE); Friedrich Wetterling, Dublin (IE); Michael Zipparo, Acme, PA (US)

(21) Appl. No.: 19/159,891

(22) PCT Filed: Feb. 28, 2024
(86) PCT No.: PCT/IB2024/051930

§ 371 Date: Aug. 26, 2025

Related U.S. Application Data
(60) Provisional application No. 63/448,924, filed on Feb. 28, 2023.

(51) Int. Cl. A61B 8/08; A61B 8/00; A61B 8/06

(52) U.S. Cl. CPC A61B 8/0891; A61B 8/06; A61B 8/4236; A61B 8/4488; A61B 8/461; A61B 8/483; A61B 8/5223; A61B 8/543

(57) ABSTRACT

Systems and methods are disclosed that provide for regular, periodic or continuous monitoring of fluid volume based on direct measurement of dimensions of a jugular vein of a patient using a sensor disposed proximate the jugular vein. Measurements of the jugular vein are made using ultrasound, which may include determination of a two or three-dimensional ultrasound image of the jugular vein. Based on the monitored changes in the jugular vein physical dimensions, information about patient fluid state across the euvolemic range of fluid states can be determined. This may provide earlier warning of hypervolemia or hypovolemia and allow for the introduction or modulation of patient treatments to permit more stable long-term fluid management.

### RELATED APPLICATION DATA

This application claims the benefit of priority of U.S. Provisional Patent Application Ser. No. 63/448,924, filed Feb. 28, 2023, and titled “Systems and Methods for Jugular Vein Measurement Using Ultrasound”, which is incorporated by reference herein in its entirety.

### FIELD OF THE DISCLOSURE

The present invention generally relates to systems and methods for patient fluid management and monitoring. In particular, the present invention is directed towards systems and methods for jugular vein measurement using ultrasound.

### BACKGROUND

Heart Failure (HF) is a systemic and chronic disease which can involve many organs, including the liver, kidneys, and lungs. Continuous monitoring of the arterial and venous function, in addition to pulmonary function provides insight into the progression of the disease and its side effects. Other volumetric-related conditions and therapies such as dialysis, ultrafiltration, pulmonary hypertension, and hypertension present a need for patient fluid volume sensing and monitoring.

A significant challenge in the treatment of acute heart failure patients is the management of the patient fluid volume. Similar challenges are also presented in the treatment of renal failure patients, and, in fact, studies have shown a direct correlation, and potentially causal relationship, between heart and renal failure conditions with respect to patient fluid management, e.g., Silverberg, et al., The association between congestive heart failure and chronic renal disease, Curr. Opin. Nephrol. Hypertens. (2004) 13:163-170. Acute heart or renal failure can lead to peripheral and pulmonary edema if not properly treated, but too aggressive of a treatment can lead to a hypovolemic state in which the patient lacks sufficient fluid volume. Treatments may include dialysis, ultrafiltration, diuretics and other drug administration. Lifestyle factors such as fluid intake and diet, specifically salt intake also may be monitored and modulated. Traditionally, diagnostic techniques used in monitoring fluid status were based on various externally observable symptoms (e.g., jugular vein distention, edema, patient weight change). Also, central venous catheterization (CVC) to monitor central venous pressure (CVP) has been used as a fluid status indicator. However, there are a number of serious risks associated with CVC, such as infection and thrombosis, and reliance on externally observable or measurable symptoms presents an obvious drawback in that the observable response to a therapy is often significantly delayed relative to acute changes in physiological status.

Monitoring fluid status can also be used as a predictor for onset of acute decompensated heart failure (ADHF), which is a significant factor driving rehospitalization of heart failure patients. There is potential to significantly reduce hospitalizations if a sufficiently early and or sensitive signal of increasing patient fluid volume can be detected. However, drawbacks of traditional diagnostic tools as mentioned above make such tools relatively ineffective as early predictors of ADHF.

In addition to heart failure patients, haemodialysis patients also have a chronic need for careful volume management. Large volumes of fluid are involved in the haemodialysis process and managing patients so that they don't end up hypovolemic or overloaded with fluid requires careful management using the currently available, sub-optimal tools.

### SUMMARY OF THE DISCLOSURE

Aspects of the present disclosure include sensors configured to monitor and measure venous geometry that can be used for a variety of applications, including for patients suffering from heart failure and patients with other volumetric-related conditions or utilizing volumetric-related therapies such as dialysis, ultrafiltration, pulmonary hypertension, and hypertension.

Systems of the present disclosure may be used to perform geometric and/or flow measurement and monitoring of the jugular vein in order to identify fluid status for management of heart failure patients. In specific embodiments, the geometric measurements could be vessel cross-sectional diameter, cross-sectional area or vessel shape, specifically ovality, and how these features change over time as they are subject to both the cardiac and respiratory cycles and or physiological patient maneuvres such as a breath hold.

The efficacy of these measurements can be further enhanced with the ability to discern the extent to which the congestion of the liver and/or kidneys contributes to the overall HF condition. Additionally, monitoring the structures and flowrates of the jugular vein, venae cavae, portal vein, renal veins and aorta provide additional detail on the function of the cardiovascular system and fluid status of a patient.

In addition to correlation with right atrial pressure, the diameter of the jugular vein may correlate with renal function and renal sodium retention, which are also very important prognostic factors of heart failure. Therefore, increasing jugular vein diameter, area or volume and/or pressure may be a very effective early indicator of worsening heart failure condition.

Use of a minimally-invasive, portable ultrasound device for vascular monitoring and intravascular volume management, which enables remote monitoring of vessel geometry and flowrates without the requirement for a trained operator, is desirable. Systems and sensors of the present disclosure provide for measuring, monitoring and modeling of the vascular system to identify structures and overcome the challenge of monitor positioning accuracy.

Standard external ultrasound monitoring requires a skilled user to initiate and perform the reading. Subcutaneous and transdermal systems described herein provide the advantage of not requiring user input to initiate a reading. These systems may initiate readings via an automated trigger, which can be based on time, on intermittent signals obtained in low power mode, or on input from an onboard accelerometer.

Sensors of the present disclosure enable multiple measurements for minimally-invasive estimation of the volumes of blood in different areas. For example, the jugular vein area, diameter, and collapsibility with cardiac and respiratory cycles provide an indication of intravascular volume. Jugular vein geometric response to perturbations including maneuvers such as breath hold, stand up from seated and others may provide information relating to the splanchnic activation or vascular tone as well as vascular volume. The portal vein diameter and flow rate could provide an indication of splanchnic activation and volume. The renal flow rates could provide an indication of renal function. All of these factors are useful in the diagnosis and treatment of vascular volume conditions.

Aspects of the present disclosure also include monitors that can provide immediate feedback on a haemodialysis patient's volume status before, during and after haemodialysis sessions.

Embodiments disclosed herein include a system for managing patient body fluid volume, comprising in some examples a wireless sensing device, a sensing device control module and a patient management system. The wireless sensing device is configured to be positioned proximate to a patient's jugular vein to measure a physical dimension of the jugular vein and generate a measurement signal indicative of the measured jugular vein physical dimensions. The sensing device control module is configured to wirelessly communicate with the sensing device to at least receive the measurement signal from the sensing device and in some embodiments also provide the ability to wirelessly charge the sensing device. In some examples a mobile computing device such as a phone, tablet, or laptop with appropriate software may be used as the control module. The patient management system may include a processor and memory, and is configured to receive or generate measurement data representative of the measured jugular vein physical dimension based on the measurement signal, to receive patient specific information, and to execute instructions contained in the memory responsive to received patient specific information and the measurement data. The instructions contained in memory may include instructions to prompt for initiation of a sensor measurement of the jugular vein physical dimension and generate an alert signal when the measured jugular vein physical dimension is outside predetermined clinical limits for the patient. The instructions may further include instructions to wirelessly communicate with the sensing device to monitor changes in the measured jugular vein dimension throughout an euvolemic region defined for the patient. In some embodiments, the physical dimension of the patient's jugular vein is at least one of jugular vein diameter or jugular vein area. In other embodiments the changes of these features over the cardiac and respiratory cycles and or patient maneuvres are the inputs into the diagnostic/therapeutic algorithm.

In some examples the present disclosure also includes a system for managing patient body fluid volume that includes a processor and memory configured to communicate with the wireless sensing device and the sensing device control module. The processor may be configured to receive measurement data representing physical dimension measurements of a jugular vein by the sensing device and execute instructions contained in the memory responsive to the measurement data. The processor also may be configured to receive patient specific information. The instructions stored in memory may comprise defining euvolemic, hypovolemic and hypervolemic regions for the patient wherein the euvolemic range, the hypovolemic range and the hypervolemic range are correlated to jugular vein diameter or volume measurements for the patient, identifying a hypovolemic warning zone for the patient encompassing a portion of the defined euvolemic region at a lower end of the euvolemic region adjacent the hypovolemic region, identifying a hypervolemic warning zone for the patient encompassing a portion of the defined euvolemic region at an upper end of the euvolemic region adjacent the hypervolemic region, and generating an alert signal when measured jugular vein diameter or volume falls within either of the hypovolemic warning zone or hypervolemic warning zone of the defined euvolemic region before the patient fluid state reaches one of the hypovolemic region or hypervolemic region, respectively.

In some embodiments the wireless sensing device may comprise an ultrasound transducer and anchor element configured to anchor the ultrasound transducer proximate the jugular vein in a fixed position relative to the jugular vein wall. In some embodiments this anchor element is specifically designed to prevent rotation of the sensing element. In some examples the wireless sensing device is attached or adhered to the patient's skin. In other examples, the wireless sensing device is subcutaneously implanted adjacent the jugular vein.

Some of the embodiments disclosed herein describe the placement of the sensing device in the subcutaneous space proximate to a vein of interest. In some examples a custom-designed insertion tool may be utilized to facilitate the accurate deployment of these devices.

Examples of the present disclosure may also include methods of managing patient body fluid volume. One embodiment of the disclosed methods may include directly measuring a physical dimension of the patient's jugular vein with a sensing device disposed proximate the jugular vein, wirelessly communicating with the sensing device to monitor changes in the measured jugular vein dimension throughout an euvolemic region defined for said patient, and generating an alert signal when measured jugular vein diameter or area approaches or falls outside predetermined clinical limits for the patient. The alert signal may be direct to a healthcare provider or to the patient. When directed to the patient, the alert signal may include a prompt to initiate a patient self-directed treatment or medication change.

In some embodiments the predetermined clinical limits may include at least a first, lower limit indicative of the patient fluid state trending towards hypovolemia, and a second, higher limit indicative of the patient fluid state trending towards hypervolemia. Further, each of the predetermined limits may fall within the euvolemic range defined for the patient. In such embodiments, further method steps may comprise setting (1) a hypovolemic warning zone for the patient encompassing a portion of the defined euvolemic region at the lower end of the euvolemic region adjacent a hypovolemic region and (2) a hypervolemic warning zone for the patient encompassing a portion of the defined euvolemic region at the upper end of the euvolemic region adjacent a hypervolemic region, wherein the euvolemic range, hypovolemic range and hypervolemic range are correlated to jugular vein physical dimension measurements for the patient.

Some examples are: a diagnostic method for monitoring patient body fluid volume comprises positioning a sensing device proximate to a patient's jugular vein, wherein the sensing device is configured to measure at least one physical dimension of the jugular vein; wirelessly monitoring changes in measured jugular vein physical dimension over time throughout an euvolemic region defined for said patient; identifying (1) a hypovolemic warning zone for the patient encompassing a portion of the defined euvolemic region at the lower end of the euvolemic region adjacent a hypovolemic region and (2) a hypervolemic warning zone for the patient encompassing a portion of the defined euvolemic region at the upper end of the euvolemic region adjacent a hypervolemic region, wherein the euvolemic range, hypovolemic range and hypervolemic range are correlated to jugular vein physical dimension measurements for the patient. Another step in such a method as disclosed may include signaling a warning state when measured jugular vein physical dimension falls within either of the hypovolemic warning zone or hypervolemic warning zone of the defined euvolemic region before the patient fluid state reaches one of the hypovolemic region or hypervolemic region, respectively.

In some examples a plurality of 2D ultrasound images may be obtained by a body-worn ultrasound transducer array. The plurality of 2D ultrasound images may be obtained from a plurality of directions or locations on the body, by a body-worn ultrasound transducer array or other form of ultrasound transducer. The plurality of 2D ultrasound images may be obtained using beam forming. Performing image analysis on a 2D ultrasound image to identify the jugular relative to other anatomical structure may be achieved via processes similar to those disclosed in WO2023/037017 Vascular Imaging and Measurement Using Ultrasound, and may include:
identifying at least one anatomical structure such as the carotid artery or other known structures proximate the jugular vein;
identifying the jugular vein in the image by its known position relative to the identified anatomical structure;
identifying at least one anatomical landmark in the image may comprise the use of Doppler data to identify the direction and velocity of blood flow;
identifying at least one anatomical structure in the image may comprise the use of known geometric properties of the at least one anatomic structure; and
performing image analysis on the ultrasound image to identify the jugular vein relative to other anatomical structures may comprise identifying anatomical structures using edge detection.

The method may further comprise:
verifying the identification of the jugular vein in the image using an additional metric selected from a group comprising Doppler velocity, direction of blood flow, pulsatility, distances, pulmonary B-line measurements and audio respiratory data or vessel response to a patient manuevre;
determining the cross-sectional area of the jugular vein from the cross-sectional image of the jugular vein in the selected slice may comprise using edge detection techniques to identify the wall of the jugular vein and analysis and combination of measures from multiple transducers;
determining the cross-sectional area of the jugular vein from the cross-sectional image of the jugular vein in the selected slice may comprise the fitting of an ellipse to the jugular vein; and
determining the cross-sectional area of the jugular vein from the cross-sectional image of the jugular vein in the selected slice may comprise the use of thresholding techniques.

The method may further comprise the use of blob detection and analysis techniques to determine the cross-sectional area of the jugular vein from the cross-sectional image of the jugular vein in the selected slice.

The steps of providing, performing, selecting and determining may be repeated over time to obtain a series of measurements.

Throughout the series of measurements, each slice selected may comprise the same cross-sectional image of the jugular vein.

The selected single slice may differ between subsequent measurement time points in the series of measurements due to movement of the jugular vein during respiration.

The method may further comprise plotting the series of measurements against time in a graph.

The method may further comprise determining at least one of minimum area, mean area, maximum area and collapsibility of the jugular vein from the ultrasounds data and/or the graph.

The method may further comprise comparing at least one of the determined minimum area, mean area, maximum area and collapsibility of the jugular vein to a threshold to record an event.

The series of measurements may be analysed to identify trends or fluctuations against an allowable tolerance over a period of time.

Determining the cross-sectional area of the jugular vein from the cross-sectional image of the jugular vein in the selected slice may comprise the use of a neural network to mark the jugular vein in the ultrasound image.

The disclosure further provides a computer program product comprising instructions which, when the program is executed by a computer, cause the computer to carry out the aforementioned method.

The disclosure further provides a computer-readable storage medium comprising instructions which, when executed by a computer, cause the computer to carry out the aforementioned method.

The ultrasound images may be provided by a portable ultrasound device comprising: an ultrasound transducer comprising a 1D or 2D array of independently controllable ultrasound transducer elements for producing an ultrasonic pulse; a power source for providing an electrical signal to the ultrasound transducers, which may or may not include a charging capability; and analysis means comprising: a beam former for optimizing transmitted ultrasound signals; a system for processing received ultrasonic signals; and a communications interface for communicating data for subsequent analysis.

Each transducer element may comprise at least one piezoelectric element. Alternatively, each transducer element may comprise at least one capacitive micromachined ultrasonic transducer (CMUT). The ultrasound transducer may be configured for at least one of B-mode ultrasonography and Doppler ultrasonography.

The data communicated may comprise a plurality of 2D scans.

The analysis means may be configured to compile a 3D representation from a plurality of 2D scans. The data communicated may then be assembled to provide the 3D representation or image stack, including by processes similar to those disclosed in WO2023/037017 Vascular Imaging and Measurement Using Ultrasound.

The ultrasound device may perform imaging of B-lines of anatomical structures. In some examples the ultrasound transducer may comprise a 1D array of transducers.

Systems of the disclosure further comprise display and communication means. The ultrasound system may further comprise a user interface. The user interface may permit manual activation of the device. This permits the user to self-activate the device when positioned and may also provide guidance or feedback to the user, based on automated analysis and identification of anatomical structures in the imaged region, on location and angulation of the device to provide complete imaging of the intended region of interest. The ultrasound device may further comprise a communications module.

Preferably, the ultrasound transducer is generally rectangular shaped in cross-section with an elongated rectangular shape in plan and elevation views. Other embodiments have a curved shape in at least one elevation view to align the ultrasound transducers around a central origin roughly approximating the centre of the target vessel. The ultrasound transducer may comprise a length of 20mm-100 mm with a width and height of 5mm-50 mm.

Securement means for securing the ultrasound transducer on the neck above the jugular vein in one or more predetermined positions may be provided. The securement means may comprise adhesive securement means. Such securement means may also provide the medium for the transfer of ultrasound vibrations between the transducer and the body. The securement means may alternatively or additionally comprise at least one strap for strapping the ultrasound transducer to the neck. The securement means may additionally or alternatively comprise a subcutaneous magnetic coupling and corresponding magnetic housing coupling to enable consistent device placement. The securement means may additionally or alternatively comprise anchor features for subcutaneous implantation of the ultrasound sensor.

Such securement means may include a housing to facilitate the repeated attachment and removal of the transducer from the securement means or patch while also providing a method to maintain acoustic coupling between the transducer and the skin.

The body may additionally be marked by way of tattoo or similar to aid the user in correctly positioning the sensor. In one embodiment the adhesive securement means may comprise means for marking the skin, such as a die or temporary tattoo, to facilitate repeat placement. In another embodiment magnetic elements could be permanently implanted, sub-dermally, in the patient. These elements would then align and attach to corresponding, magnetic elements in the ultrasound device, thus repeatedly positioning it accurately. The processing means may be configured to compensate for sensor placement error.

The portable ultrasound device may further comprise means for tracking the motion and position of the patient, for example by way of an accelerometer or gyroscope.

The present disclosure further provides a method for vascular monitoring and intravascular volume management.

A device for monitoring a blood vessel of a patient is provided that includes a housing, an array of ultrasonic transducers attached to the housing, and a microcontroller in the housing connected to the array of ultrasonic transducers. The device is configured to be implanted subcutaneously in the patient.

Additionally or alternatively, the array of ultrasonic transducers is arranged such that, when the device is positioned proximate the blood vessel, an aggregate ultrasonic image plane that encompasses a cross section of the blood vessel is scannable by the array of ultrasonic transducers.

A method of measuring dimensional parameters of a blood vessel of a patient is provided that includes positioning a sensor subcutaneously proximate the blood vessel of the patient, transmitting ultrasonic waves from the sensor toward the blood vessel, receiving reflected ultrasonic waves from the blood vessel at the sensor; and determining a plurality of blood vessel parameters based on the received reflected ultrasonic waves.

Additionally or alternatively, the plurality of blood vessel parameters includes at least one of an area and a diameter of the blood vessel

A device for monitoring a blood vessel is provided that includes a housing, an array of ultrasonic transducers attached to the housing, and a microcontroller in the housing connected to the array of ultrasonic transducers. The array of ultrasonic transducers is arranged such that, when the device is positioned proximate the blood vessel, an aggregate ultrasonic image plane that encompasses a cross section of the blood vessel is scannable by the array of ultrasonic transducers.

Additionally or alternatively, the device is configured to be implanted subcutaneously.

Additionally or alternatively, the array of ultrasonic transducers includes a plurality of transducer elements, wherein each of the plurality of transducer elements is attached to a first side of the housing, and wherein the first side of the housing is curved in one plane.

Additionally or alternatively, the array of ultrasonic transducers includes a plurality of transducer elements, each of the plurality of transducer elements is attached to a first side of the housing, and one or more of the plurality of transducer elements is positioned at an acute angle with respect to a plane of the first side of the housing.

Additionally or alternatively, the array of ultrasonic transducers includes a plurality of transducer elements, wherein each of the plurality of transducer elements is attached to a first side of the housing, each of the plurality of transducer elements is configured to emit a beam profile having a narrowed portion, and the narrowed portion is formed at a distance from the first side of the housing such that the narrowed portion is positioned near a central longitudinal axis of the blood vessel when the device is attached to or implanted in a patient.

Additionally or alternatively, the device is configured to be attached to the skin of a patient.

Additionally or alternatively, the array of transducers includes a two dimensional grid of a plurality of transducer elements.

Additionally or alternatively, the housing includes a pad for attaching to skin, a locking plate attached to the pad, and an ultrasound transducer module containing the array of ultrasonic transducers, wherein the locking plate is configured to removably receive the ultrasound transducer module.

Additionally or alternatively, a sensing electrode is included on the housing configured to detect sympathetic nervous signals.

Additionally or alternatively, a pair of electrodes is included on the housing configured to detect tone by impedance between the pair of electrodes.

Additionally or alternatively, one or more protuberances extend from the housing that are sized and configured to reduce rotation of the device within subcutaneous space.

Additionally or alternatively, the housing contains a wireless transmitter, a digital analog converter, a high voltage transmit amplifier, a multiplexer element, a transmit/receive switch, a time gain compensation amplifier, an analog digital converter, and a data buffer.

Additionally or alternatively, the microcontroller includes a module configured to process signals representative of ultrasonic waveforms and convert the signals to a plurality of dimensional blood vessel parameters.

Additionally or alternatively, the plurality of parameters include a blood vessel diameter and a blood vessel area.

Additionally or alternatively, the module is configured to process signals representative of ultrasonic waveforms to generate an image of the blood vessel.

Additionally or alternatively, the module is configured to determine a plurality of blood vessel dimensional parameters based on the image of the blood vessel.

Additionally or alternatively, the plurality of parameters include a blood vessel diameter and a blood vessel area.

Additionally or alternatively, the module is configured to determine a change in one or more of the plurality of parameters over time.

A system for monitoring a blood vessel of a patient is provided that includes a sensor including that has a housing, a wireless transmitter, an array of ultrasonic transducers attached to a first side of the housing, and a microcontroller in the housing connected to the array of ultrasonic transducers. The array of ultrasonic transducers is arranged such that, when the sensor is positioned proximate the blood vessel, an aggregate ultrasonic image plane that encompasses a cross section of the blood vessel is scannable by the array of ultrasonic transducers. A user electronic device is in communication with the wireless transmitter and includes a display with a user interface and is configured to receive data from the sensor.

Additionally or alternatively, each of the ultrasonic transducers is steerable and focusable through a width of the blood vessel.

Additionally or alternatively, the microcontroller includes a module configured to process signals representative of ultrasonic waveforms and convert the signals to image data.

Additionally or alternatively, the microcontroller includes a module configured to process signals representative of ultrasonic waveforms and convert the signals to a plurality of dimensional blood vessel parameters.

Additionally or alternatively, the module is configured to process the image data to determine a plurality of dimensional parameters of the blood vessel, the plurality of dimensional parameters including a blood vessel diameter and a blood vessel area.

Additionally or alternatively, the module is configured to determine a change in one or more of the plurality of dimensional parameters over time.

Additionally or alternatively, the user electronic device is configured to display content based on data received from the sensor.

Additionally or alternatively, the microcontroller includes an euvolemic module configured to compare a blood vessel diameter value to a range of diameter threshold values and compare a blood vessel area value to a range of threshold area values.

Additionally or alternatively, the euvolemic module is configured to transmit a warning signal to the user electronic device when the blood vessel diameter value and/or the blood vessel area value are determined to be in ranges associated with a hypervolemic state or a hypovolemic state for the patient.

Additionally or alternatively, the sensor is configured to be implanted subcutaneously.

Additionally or alternatively, the array of ultrasonic transducers includes a plurality of transducer elements, wherein each of the plurality of transducer elements is attached to the first side of the housing, and wherein the first side of the housing is curved in one plane and, when the sensor is implanted proximate the blood vessel, the first side faces the blood vessel.

Additionally or alternatively, the array of ultrasonic transducers includes a plurality of transducer elements, each of the plurality of transducer elements is attached to the first side of the housing, and one or more of the plurality of transducer elements is positioned at an acute angle with respect to a plane of the first side of the housing, and when the sensor is implanted proximate the blood vessel, the first side faces the blood vessel.

Additionally or alternatively, the array of ultrasonic transducers includes a plurality of transducer elements, each of the plurality of transducer elements is attached to the first side of the housing, each of the plurality of transducer elements is configured to emit a beam profile having a narrowed portion, and the narrowed portion is formed at a distance from the first side of the housing such that the narrowed portion is positioned near a central longitudinal axis of the blood vessel when the device is attached to or implanted in the patient.

Additionally or alternatively, the sensor is configured to be attached to the skin of the patient.

Additionally or alternatively, The system of claim 19, wherein the array of transducers includes a two dimensional grid of a plurality of transducer elements.

Additionally or alternatively, the housing includes a pad for attaching to skin, a locking plate attached to the pad, and an ultrasound transducer module containing the array of ultrasonic transducers, and the locking plate is configured to removably receive the ultrasound transducer module.

Additionally or alternatively, the sensor includes a sensing electrode on the housing configured to detect sympathetic nervous signals.

Additionally or alternatively, the sensor includes a pair of electrodes on the housing configured to detect tone by impedance between the pair of electrodes.

Additionally or alternatively, the sensor includes one or more protuberances extending from the housing and sized and configured to reduce rotation within subcutaneous space.

Additionally or alternatively, the housing contains a digital analog converter, a high voltage transmit amplifier, a multiplexer element, a transmit/receive switch, a time gain compensation amplifier, an analog digital converter, and a data buffer.

Additionally or alternatively, the microcontroller includes a comparison module configured to compare the blood vessel area to a range of threshold area values for the patient and the blood vessel diameter to a range of threshold diameter values for the patient.

Additionally or alternatively, the comparison module is configured to send instructions to send a warning to the user electronic device if the area is determined to be within a hypervolemic range of threshold area values, if the diameter is determined to be within a hypervolemic range of threshold diameter values, if the area is determined to be within a hypovolemic range of threshold area values, or if the diameter is determined to be within a hypovolemic range of threshold diameter values.

Additionally or alternatively, the comparison module is configured to determine whether the patient is in a hypovolemic state, a euvolemic state, or a hypervolemic state based on the area and the diameter.

Additionally or alternatively, the warning includes a prompt to initiate a treatment or medication change based on whether the patient is determined to be in a hypovolemic state, a euvolemic state, or a hypervolemic state.

Additionally or alternatively, the warning is an audible warning emanating from the sensor.

Additionally or alternatively, the warning is an audible warning emanating from an electronic device external to the patient.

Additionally or alternatively, when the patient is determined to be in the hypovolemic state, the treatment or medication change includes a recommendation for administration of fluids or vasodilating drugs.

Additionally or alternatively, when the patient is determined to be in the hypovolemic state, the treatment or medication change includes a recommendation for dialysis or administration or treatment guidance for diuretics or vasodilating drugs.

A method of measuring dimensional parameters of a blood vessel of a patient is provided that includes positioning a sensor proximate the blood vessel of the patient, wherein the sensor includes a housing, a wireless transmitter, an array of ultrasonic transducers attached to a first side of the housing, wherein the first side of the housing includes a length, a microcontroller in the housing connected to the array of ultrasonic transducers, wherein the array of ultrasonic transducers is arranged such that, when the sensor is implanted, an aggregate ultrasonic image plane is scannable by the array of ultrasonic transducers and encompasses a cross section of the blood vessel.

Ultrasonic waves are transmitted from the array toward the blood vessel, the reflected ultrasonic waves are received from the blood vessel at the sensor. One or more blood vessel parameters are determined from the reflected ultrasound waves, such as an area and a diameter Additionally or alternatively, the plurality of detected waveforms are processed to generate an image of the blood vessel based on the received reflected ultrasonic waves. A plurality of blood vessel parameters are determined based on the image, including an area and a diameter, and are compared to a range of threshold area values for the patient and to a range of threshold diameter values for the patient. A warning is sent to a user interface if the area is determined to be within a hypervolemic range of threshold area values, if the diameter is determined to be within a hypervolemic range of threshold diameter values, if the area is determined to be within a hypovolemic range of threshold area values, or if the diameter is determined to be within a hypovolemic range of threshold diameter values.

Additionally or alternatively, whether the patient is in a hypovolemic state, a euvolemic state, or a hypervolemic state is determined based on the area and the diameter.

Additionally or alternatively, the warning includes a prompt to initiate a treatment or medication change based on whether the patient is determined to be in the hypovolemic state, the euvolemic state, or the hypervolemic state.

Additionally or alternatively, when the patient is determined to be in the hypovolemic state, the treatment or medication change includes a recommendation for administration of fluids or vaso-constricting drugs.

Additionally or alternatively, when the patient is determined to be in the hypovolemic state, the treatment or medication change includes a recommendation for dialysis or administration or treatment guidance for diuretics or vasodilating drugs.

Additionally or alternatively, the array of ultrasonic transducers includes a plurality of transducer elements and wherein each of the plurality of transducer elements is attached to the first side of the housing, further including steering and focusing each of the plurality of transducer elements independently such that an aggregate image plane encompasses an entire cross section of the blood vessel.

Additionally or alternatively, the positioning of the sensor includes attaching the sensor to the skin of the patient.

Additionally or alternatively, the positioning of the sensor includes implanting the sensor subcutaneously.

Additionally or alternatively, the sensor is implanted subcutaneously such that a portion of the first side of sensor is in contact with the blood vessel.

Additionally or alternatively, a set of current values for the plurality of blood vessel parameters is compared to a set of previously determined values for the plurality of blood vessel parameters and a set of value changes is determined based on the comparison.

The present disclosure further provides systems and methods for marking ultrasound images, including but not limited to those obtained using the wearable or implantable ultrasound devices described herein.

### BRIEF DESCRIPTION OF THE DRAWINGS

For the purpose of illustrating the disclosed embodiments, the drawings show aspects thereof. It is to be understood, however, that the teachings of the present disclosure are not limited to the precise arrangements and instrumentalities shown in the drawings, wherein:

FIG. 1A is a schematic overview of an example system of the present disclosure for monitoring a physical dimension of a jugular vein and providing treatments and guidance to a patient in response to the measurements;

FIG. 1B is a schematic overview of another example system of the present disclosure for monitoring a physical dimension of a jugular vein and providing closed loop treatments and guidance to a patient in response to the measurements;

FIG. 2 is an exploded perspective view of an ultrasound sensor with a two-dimensional array of transducer elements in accordance with an embodiment of the present disclosure;

FIG. 3 is a side view of a wearable sensor that includes a removeable electronics module, a disposable adhesive pad, and a mechanical locking plate in accordance with an embodiment of the present disclosure;

FIG. 4 is a side view of a wearable sensor that includes a unitary electronics module and adhesive pad with gel layer in accordance with an embodiment of the present disclosure;

FIG. 5A is a plan view of a wearable ultrasound patch positioned in relation to a vessel to be measured/monitored in accordance with an embodiment of the present disclosure;

FIG. 5B is a cross-sectional view of the wearable ultrasound patch of FIG. 5A;

FIG. 6 is a cross-sectional view of another ultrasound patch disposed on a patient's neck that also illustrates ultrasonic waves and a jugular vein located below the skin;

FIG. 7 conceptually illustrates ultrasonic waves emitted by a sensor and waves reflected by the jugular vein;

FIG. 8 depicts a subcutaneous wireless ultrasound sensor in accordance with another embodiment of the present disclosure and illustrates ultrasonic waves and a jugular vein;

FIGS. 9A-9F illustrate examples of locations and orientations of ultrasound sensor arrays and various arrangements of the ultrasound elements and housing with respect to a targeted vessel;

FIGS. 10A and 10B are illustrations depicting ultrasound scans showing a jugular vein location 1-2 cm below a patient's skin;

FIGS. 11A, 11B, and 12 illustrate the general location of the jugular vein in a patient;

FIG. 13 illustrates an example placement of a sensor patch on the skin over the jugular vein for monitoring a diameter or other physical dimension of the vein over time;

FIG. 14 illustrates a placement of the transducer array relative to the jugular vein such that beams of at least two transducers will be directed at the vein even if the position of the vein changes;

FIG. 15A conceptually illustrates an ultrasound transducer array and vein arrangement in space in which the vein is compressed;

FIG. 15B depicts a temporal signal recording of reflected waves received at transducer elements for the compressed vein shown in FIG. 15A;

FIG. 15C conceptually illustrates an ultrasound transducer array and vein arrangement in space in which the vein is inflated;

FIG. 15D depicts a temporal signal recording of reflected waves received at transducer elements for the inflated vein shown in FIG. 15C;

FIG. 16 is a functional block diagram of an ultrasound sensor and associated analog and digital hardware for processing echoes and transmitting information externally in accordance with an embodiment of the present disclosure;

FIG. 17 is a functional block diagram of a system in accordance with another embodiment of the present disclosure including an ultrasound sensor and associated analog and digital hardware for processing echoes and transmitting information to an external device;

FIG. 18A schematically illustrates a single transducer element for a sensor of the present disclosure;

FIGS. 18B and 18C illustrate an example of a one-dimensional array of transducer elements for a sensor of the present disclosure;

FIG. 18D illustrates a sensor with the array of transducers of FIG. 18B positioned with respect to a vein and shows an example ultrasonic beam profile;

FIG. 19A depicts another example of a one-dimensional array of transducer elements for a sensor of the present disclosure;

FIG. 19B illustrates the transducer array of FIG. 19A positioned with respect to a vein; and

FIG. 20 is a block diagram of a computing system that can be used to implement any one or more of the methodologies disclosed herein and any one or more portions thereof.

### DETAILED DESCRIPTION

Embodiments disclosed herein include systems and methodologies allowing for regular, periodic or continuous monitoring of fluid volume of a patient more accurately than current techniques and with reduced lag time before changes in volume status are observed, thus providing earlier warning of hypervolemia or hypovolemia and enabling the modulation of patient treatments to permit more stable long term fluid management. Further, in acute situations, the methods and systems disclosed enable more rapid reduction of excessive intravascular volume and edema and restoration of more ideal fluid balance, with lessened risk of creating a hypovolemic state as can be created when patient “drying” treatments overshoot due to the response of patient monitoring devices or protocols.

A challenge presented for managing patient fluid balance for patients in decompensated heart failure is bringing down excess fluid volume as quickly as possible without overshooting and taking the patient into a potentially equally dangerous hypovolemic state or damaging their kidney function. In the longer term management of heart failure, maintaining fluid balance is still a challenge, but in this case it involves maintaining the patient well within a safe fluid state without unintentionally migrating into a hypervolemic or hypovolemic state.

“Euvolemia” refers to the normal fluid volume in a patient's body, and “euvolemic region” refers to a range of fluid volume within the patient that is clinically characterizable as normal or not requiring intervention. (“Euvolemia” is also sometimes referred to in the medical literature as “normovolemia.”) The euvolemic region, as explained in more detail below, encompasses a fluid state or volume range across which measurement of central venous pressure (CVP) in the jugular vein is generally non-responsive to changes in fluid volume. “Hypervolemia” refers to a state in which a patient's body fluid volume exceeds a normal range, and “hypervolemic range” refers to a range of fluid volume within the patient that is clinically characterizable as excessive. Intervention may be indicated when a patient trends towards, enters into or persists within the hypervolemic range. “Hypovolemia” refers to a state in which a patient's body fluid volume is below a normal range, and “hypovolemic range” refers to a range of fluid volume within the patient that is clinically characterizable as insufficient. As with the hypervolemic range, intervention also may be indicated when a patient trends towards, enters into or persists within the hypovolemic range. These body fluid states are not static nor are they uniform in terms of absolute volume. While it is possible to assign approximate ranges of parameters generally corresponding to the different fluid states, it can be difficult in practice, with existing diagnostic tools and methods, to identify where a particular patient's fluid state may reside with respect to the euvolemic, hypovolemic and hypervolemic ranges, and it is not currently possible for this data to be obtained, daily from patients homes without the presence of a skilled operator.

In further alternative embodiments disclosed herein, patient fluid state can be further modulated based on a combination of jugular vein data with other monitoring signals; symptoms and clinical input, by use of jugular vein data as it is influenced by some stimulus (e.g., exercise; leg raises, Valsalva maneuver) to indicate either system capacitance or redistribution of fluid, by use of jugular vein measurements from a sensor proximate the jugular vein to transmit regular information locally to help the clinical management of patients, e.g. patients managing their own dialysis and/or diuresis at home, or by use of jugular vein measurement from a vein dimension measurement sensor to control drug delivery (e.g., like a closed loop implanted system for diabetes). Advantages achievable with disclosed systems and methods may include improved reduction of excessive intravascular blood volume in the clinical setting, through the controlled use of diuretics, more accurate management of blood volume in the home setting, through the monitoring of patients and use of a treatment algorithm, more rapid dialysis through the monitoring of volume and informed variation of dialysis rate.

Based on systems and methods disclosed herein, a patient healthcare provider can devise defined early warning zones for a hypovolemic region (OE) and hypervolemic region (RE). Just as the euvolemic region boundaries vary from patient to patient based on many physical and health related factors, such as age, sex, obesity and disease state, the hypovolemic region and hypervolemic region boundaries may vary by patient as well. The early warning zones reside within the euvolemic range immediately adjacent the hypovolemic and hypervolemic regions such that the patient may still be considered to be within acceptable fluid balance parameters when in the early warning zones. However, the ability to define early warning zones as such based on jugular vein diameter or area measurements means that appropriate interventions may be initiated earlier, before the patient reaches higher levels of criticality, and thus fluid volume may be controlled more precisely and smoothly to minimize risk of shock from sudden interventions and/or overshoot of therapy targets.

Jugular vein diameter or area measurements also may be used in combination with other diagnostic signals to provide guidance on therapeutic intervention, e.g. diuretics versus vasodilators. When used with intervention, the jugular vein diameter or area measurement time dynamic response may be used to give information on the fluid status/distribution of the patient to guide therapy intervention. Response of jugular vein diameter or area measurements to a perturbation, e.g., physical activity, can cause sympathetic nerve response and fluid redistribution. Monitoring changes in jugular vein diameter or area will thus provide information on fluid volume status. In other words, an act as simple as a leg raise may cause a fluid change/redistribution that could also provide information on fluid volume status that would not be visible with pressure-based systems. Thus, in certain embodiments, at-risk patients may have continuous or near-continuous monitoring of jugular vein diameter or area changes during physical activity.

Sensed changes in jugular vein diameter or area also may be combined with other parameters such as with a brain natriuretic peptide test (BNP) or pressure/edema signals to help guide therapy intervention or differentiate patient phenotype (e.g. heart failure with reduced ejection fraction (HFrEF) v. heart failure with preserved ejection fraction (HFpEF)). Examples include detection of low collapsibility plus peripheral edema as an indication for diuretic therapy or detection of low collapsibility without peripheral edema as an indication for vasodilator therapy. Combination of monitoring jugular vein diameter or area changes with implanted pressure-based monitors (in the inferior vena cava (IVC), right atrium, right ventricle, pulmonary artery, left atrium, or other vessel) also may permit determination of abdominal pressure and flow in the jugular or IVC. In addition, the jugular vein monitoring devices of the present disclosure may include additional sensors to measure additional parameters within the jugular vein such as blood flow rate and venous pressure. Further, measurement of the dimensions or additional parameters of other vessels, such as the superior vena cava, pulmonary artery, or heart chambers, may in some cases be advantageous to supplement jugular vein measurement. In such cases, dimensional measurement devices similar to the jugular vein monitoring device of the present disclosure may be configured for implantation or positioning proximate such other vessels. In such embodiments, the methods and systems of the present disclosure may be adapted to receive such supplementary data from these sources and incorporate such data in the determination of fluid status, heart failure status, appropriate thresholds for communicating alerts or messages, or therapeutic treatment plans or recommendations.

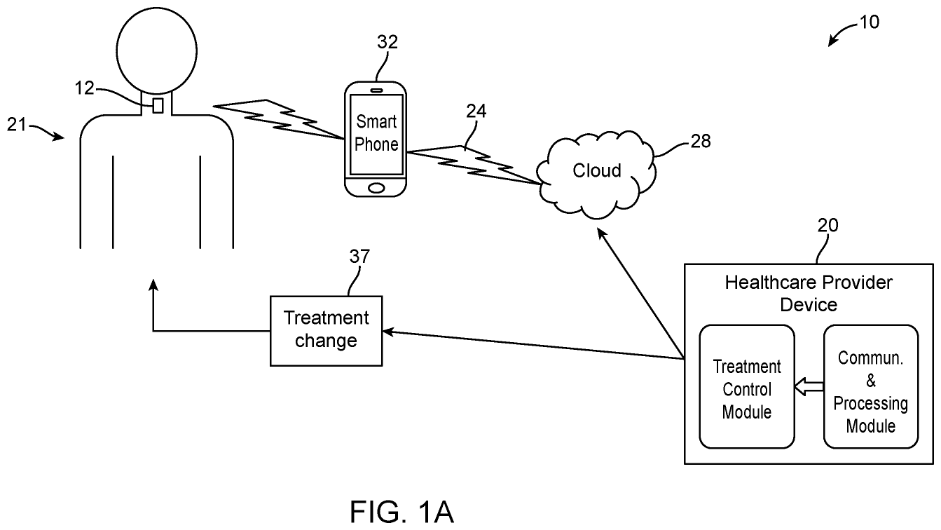

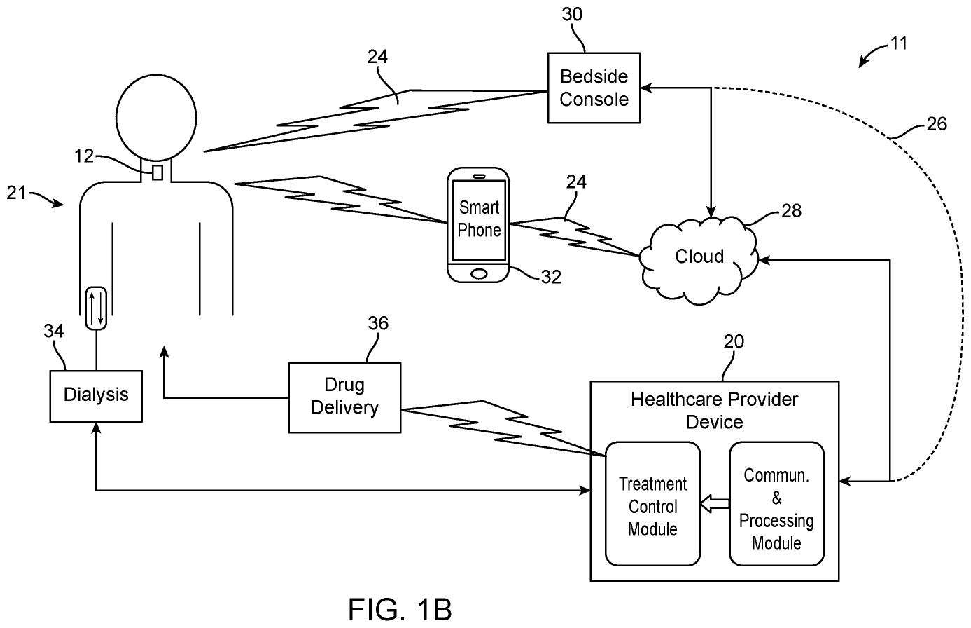

FIG. 1A schematically represents one embodiment of a system 10 including a jugular vein diameter/area measurement monitoring device 12 positioned at a monitoring location proximate the jugular vein of a patient 21. In the example illustrated, monitoring device 12 is an ultrasound-based device implanted subcutaneously proximate the jugular vein and uses an ultrasound signal reflected off the walls of the jugular vein to detect a distance to the walls of the vein by measuring the time-of-travel of the signal from the device to the wall of the vein and back to the device. As described more below, two opposing walls of the vein can be identified and a distance between the walls determined, which corresponds to a vein diameter measurement.

Measurements of jugular vein diameter or area by monitoring device 12 may be made continuously or for periods covering one or more respiratory cycles to determine the variation in jugular vein dimensions over this cycle. Further, these measurement periods may be taken continuously, at preselected intervals, and/or in response to a remotely provided prompt from a healthcare provider or patient.

Monitoring device 12 may be configured to communicate via wireless or wired connection with a smart phone 32, or other external communication/control device. Data collected by the monitoring device may be communicated ultimately to a healthcare provider device 20 via wired and/or wireless 24 communications and/or directly through hard-wired links such as telephone or local area networks or through the internet or cloud-based systems 28. Communications may be facilitated in a home or clinical treatment location or, particularly in the case of implanted monitoring devices, through mobile device 32. Healthcare provider device 20 may be configured with appropriate user interface, processing and communications modules for data input and handling, communications and processing, as well as treatment and control modules, which may include treatment algorithms as described herein for determining treatment protocols based on collected jugular vein diameter or area measurements, and systems for automated remote control of treatment devices based on determined treatment protocols as elsewhere described herein. Examples of such treatment changes 37 include, but are not limited to, medication changes or device therapies. Examples of treatments include, when measured dimensions correspond to values that fall within the hypovolemic warning zone, administration of fluids or vaso-constricting drugs, and when measured dimensions correspond to values that fall within the hypervolemic warning zone, dialysis or administration or treatment guidance for diuretics or vasodilating drugs.

FIG. 1B schematically illustrates an alternative exemplary system 11 including a jugular vein diameter/area measurement monitoring device 12 positioned at a monitoring location proximate the jugular vein. In the example illustrated, monitoring device 12 is an ultrasound-based device anchored proximate the jugular vein and uses an ultrasound signal reflected off the walls of the jugular vein to detect a distance to the walls of the vein by measuring the time-of-travel of the signal between the walls and the device. As described more below, the two opposite walls of the vein can be identified and a distance between the walls determined, which corresponds to a vein diameter measurement (i.e., the maximum distance between the walls).

Measurements of jugular vein diameter or area by monitoring device 12 may be made continuously or over one or more respiratory cycles to determine the variation in jugular vein dimensions over this cycle. Further, these measurement periods may be taken continuously, at preselected intervals and/or in response to a remotely provided prompt from a healthcare provider or patient.

Monitoring device 12 may be configured to communicate via wireless or wired connection with a bedside console 30, smart phone 32, or other external communication/control device. Data collected by the monitoring device may be communicated ultimately to a healthcare provider device 20 via wired and/or wireless 24 communications and/or directly through hard wired links such as telephone or local area networks 26 or through internet or cloud-based systems 28. Communications may be facilitated by bedside console 30 in a home or clinical treatment location or, particularly in the case of implanted monitoring devices, through a mobile device 32, such as a smart phone. Healthcare provider device 20 may be configured with appropriate user interface, processing and communications modules for data input and handling, communications and processing, as well as treatment and control modules, which may include treatment algorithms as described herein for determining treatment protocols based on collected jugular vein diameter or area measurements, and systems for automated remote control of treatment devices based on determined treatment protocols as elsewhere described herein. Examples of such treatment devices include, but are not limited to, dialysis machine 34 and drug delivery device 36. Examples of treatments include, when measured dimensions correspond to values that fall within the hypovolemic warning zone, administration of fluids or vaso-constricting drugs or slowing of dialysis process, and when measured dimensions correspond to values that fall within the hypervolemic warning zone, dialysis or administration of diuretics or vasodilating drugs or acceleration of dialysis process. Warning signals may be sent when a patient's fluid state is determined to be in a warning zone, and may include audible signals or directions which may come from the attached or implanted sensor and/or the external electronic device in communication with the sensor.

Jugular vein physical dimension data and/or fluid volume state information derived therefrom may also be communicated directly to the patients themselves, along with therapy advice based on this data, using pre-determined algorithms/implanted medical devices. Communications protocols throughout the system may include bidirectional communications to permit a healthcare provider (or other appropriately trained operator at another point in the system) to alter overall monitoring protocols executed at the monitoring device or, for example, to request additional queries by the monitoring device outside the current operational protocol.

Other embodiments include systems for patient self-directed therapy, for example with jugular vein volume metrics data utilized directly by the patient with or without clinician overview, e.g., for self-administration of drugs or other therapies. Such systems may also be implemented for home dialysis and/or peritoneal dialysis or home-based patient self-management of heart failure. In an example, wireless communication between the jugular vein monitor and the patient's cell phone or computer allows for continuous or periodic transmission of jugular vein data and the use of software applications to provide alarms or reminders, graphically present trends, suggest patient actions, drug dosage options, or treatment system settings, and allow communication with physicians.

Other than the function of the jugular vein dimension measurement sensor 12 itself, functions of system 11 may be distributed among functional elements in any number of arrangements as best suited to a home or clinical application and the intended location of sensor reading function, e.g., in a home or hospital setting. For example, all system functions (except sensor specific functions as mentioned) may be contained in a single functional unit in the form of a stand-alone patient management system. Alternatively, functions may be distributed among mobile devices networked with secure cloud computing solutions. For example, the jugular vein dimension measurement sensor may communicate directly with a patient-owned smart phone to receive signals indicating jugular vein physical dimension measurements and, in turn, wirelessly transmit those signals, e.g., via Wi-Fi or cell network, etc., to a remote computing system such as a cloud-based system for distribution to further mobile devices in the possession of healthcare providers. Hand-held devices such as tablets or smart phones may communicate directly with controlled treatment delivery devices, or such devices may be controlled by a self-contained patient management system. Further, processing necessary for operation of the system also may be distributed or centralized as appropriate, or may be duplicated in multiple devices to provide safety and redundancy. As just one example, as shown in FIGS. 1A and 1B, both bedside console 30 and smart phone 32 may be capable of performing identical functions and communicating with healthcare provider device 20 to report results of execution of the assigned functions. Thus, the specific arrangement of the components in the schematic presentations of the illustrative examples in FIGS. 1A and 1B is not to be considered as limiting with respect to possible arrangements for distribution of disclosed functions across a network.

Utilizing embodiments described herein, it is possible to determine not only jugular vein metrics associated with blood volume status, but also respiration and heart rates. New clinical work flows also may be employed based on these multiple metrics to increase opportunities for improved patient outcomes. For example, utilizing a system employing a jugular vein diameter/area monitoring device, a method may include, for example, after device implantation or installation, an initial detection algorithm that calls for periodic readings of jugular vein diameter/area when the patient is at home. Such periodic readings may, for example, be taken weekly, daily or on other appropriate periods as determined by the healthcare provider based on patient parameters such as when sleeping or when in specific body positions as determined by on-board sensors and systems. In some embodiments the reading may be taken with the patient lying supine in bed and in proximity to a bedside console. Alternatively, the jugular vein diameter/area monitoring implant may include on-board memory, in which case it may also monitor jugular vein diameter or area measurements continuously or every few minutes and record the readings over the course of a day, and transmit data once a day. Trend data for the selected period could be developed in this manner. Readings may be transmitted through the communications network as established to the clinical interface. Based on jugular vein metrics, the treatment algorithm determines necessary interventions, if any. When conditions or trends are indicated within predetermined “normal” parameters for the specific patient, no action is indicated and the system resets for the next periodic reading. However, if a condition or trend is indicated outside of the predetermined “normal” parameters, a clinical alert may be generated and suggested interventions established by the applicable treatment algorithm employed. For example, in response to a clinical alert, the healthcare provider directed care or patient self-directed care may be considered as suggested interventions and one or more effected consistent with the patient treatment plan. For patients already in a clinical setting, this may include instructions to other treatment devices connected to or working with the patient (for example, as shown in FIG. 1B with system 11). Other interventions or hospitalizations may be dictated for ambulatory patients or those otherwise outside a clinical setting when the alert is generated. Particularly for patients outside a clinical setting when an initial alert is generated, through bidirectional communication, the system allows the healthcare provider to instruct the monitoring device to generate one or more confirmatory monitoring signals before treatments are added or changed, or hospitalization required. After an intervention, the system may optionally reset for the next periodic reading. Depending on the nature or type of the initial clinical alert and interventions, patient parameters may be modified by healthcare provider input or, optionally, in some cases, automatically by the system. Modifications may include, for example, changes in frequency of prompts for periodic readings or changes in treatment algorithms that may be directed by the healthcare provider or patient self-directed.

Further exemplary embodiments may include patient fluid management methods comprising steps such as measuring the diameter of the jugular vein in a patient, calculation of jugular vein collapsibility index and/or estimating patient blood volume based on jugular vein collapsibility, applying a treatment to the patient to effect a change in patient fluid level when determined fluid level is outside predetermined limits, continuously or substantially continuously monitoring jugular vein diameter or area measurements, such as change in jugular vein diameter, during said treatment and modulating said treatment in response to monitored change in the jugular vein diameter. With such methods, treatment modulation may be accomplished in near real-time as desired. The measurement and treatment may be directly linked and operate directly in a closed loop.

Further alternative embodiments may involve monitoring jugular vein dimension variation over the respiratory and cardiac cycle, which may additionally include measurement/derivation of both breathing rate and heart rate on their own and/or in conjunction with different breathing maneuvers, e.g., breath hold, Valsalva, or exercise. Longitudinal variation over days or weeks also may be a factor monitored. In another aspect, embodiments disclosed may include algorithms that incorporate other physiologic data, such as vascular pressures, heart rate, weight, posture, exercise status, etc. and also may use data from other implanted sensors, or other external devices.

In yet another alternative, the modulating may comprise use of multiple treatment algorithms including trend analysis and reference baselines with daily or near-daily titration of medications, diet, and other therapeutic actions. Diuretic delivery also may be added with algorithms generally applicable to patient populations, or custom algorithms based on specific patient status or physiology, for example, HFpEF vs HFrEF, or renal functional status.

Another embodiment for a subcutaneously implanted monitor provides for detection of the tone of the vessel being monitored directly either via a sensing electrode placed in contact with the vessel wall to detect sympathetic nervous signals or via a pair of electrodes within the sensor itself configured to detect tone using impedance between the sensors. An alternative embodiment would achieve this tone detection by monitoring the response of the maximum jugular vein area during maneuvres, such as when a patient transitions from seated to standing. Patients that show a response to this maneuvre, where the maximum area changes slowly, may have normal sympathetic tone, while ones that do not show this response, where the maximum area remains relatively unchanged, may have overactive sympathetic tone and may benefit from vasodilator treatment.

Other exemplary embodiments include fluid management systems comprising at least one monitoring device positioned proximate to a patient's jugular vein and configured to monitor jugular vein diameter or area measurements, such as changes in the jugular vein diameter, and output a signal representative of those changes. A healthcare provider device may be configured to communicate with the monitoring device proximate the patient jugular vein and determine patient treatment protocols based on the output signal and an executable treatment algorithm. Interventional devices are included providing patient treatment or therapies controlled by the healthcare provider device based on the determined treatment protocols.

A further alternative embodiment is a dialysis or ultrafiltration management method comprising continuously measuring the diameter of the jugular vein in a patient during dialysis or ultrafiltration, estimating patient blood volume based on measured jugular vein diameter, and adjusting the rate of fluid removal to continuously optimize the patient's circulating blood volume. The measurement of the diameter may track diameter variations over the respiratory and/or cardiac cycle. With such a method, a patient's circulating blood volume may be rapidly reduced to an optimal level at the beginning of the dialysis session, and then maintained at that level throughout the session as interstitial fluid migrates into the circulatory system. Further alternatives in such a method may be used to optimize the dialysis procedure so as to maximize safety, by preventing episodes of hypovolemia, by maximizing safe fluid removal from the interstitial space over a given time period and/or long-term patient health, by safely maintaining the patient at a lower total body fluid volume than could otherwise be maintained.

In one example, a method of the present disclosure includes: placement of an ultrasound patch over the jugular vein to monitor its geometry; analysis of a sensor signal generated by the patch to determine cross-sectional geometry of the vein (for example, area and/or diameter and features derived from these metrics); analysis of data in comparison to previous results (which may be performed in processing components located in the patch, and/or in an edge computing device and/or in a cloud computing platform); communication of data to cloud (either directly or via phone or other local/edge computing device); alerting of healthcare professional (HCP) based on predefined thresholds; and communication to patient to modify treatment to avoid hospitalization.

As mentioned previously, systems according to the present disclosure may generally comprise sensor 12 configured for placement proximate a patient's jugular vein. Such sensors 12 may in some embodiments include control and communications modules, and one or more remote systems such as processing systems, user interface/displays, data storage, etc., communicating with the control and communications modules through one or more data links, preferably remote/wireless data links.

The present disclosure provides systems and methods for longitudinal temporal monitoring of the jugular vein using ultrasound to repeatedly/conveniently capture the same cross-sectional slice while the patient undergoes manoeuvres.

The images may be obtained from a body-worn or wearable ultrasound sensor patch or a subcutaneous ultrasound implant. In some examples, the ultrasound device is configured for use in the remote monitoring of heart failure patients, but it is not limited to this application as it is also suitable for use in other applications.

Example features of a sensor system of the present disclosure may include:
1. Adhesive on the device for securing the device over the targeted vein;
2. Marking on exterior of system indicating position with respect to the targeted vein;
3. Sensor array to facilitate measurement of vein geometry;
4. Battery located in sensor for powering sensor;
5. Computational capability to create, transmit and receive ultrasound signal;
6. Processing capability to perform signal processing or the received signal (for example, an algorithm to monitor signals from individual elements in the array and select which signals to use based on detection of walls of vein and their movement);
7. Audio capability to provide indication to instruct patient to initiate a reading or perform a manoeuvre at a specific time during reading;
8. Additional sensors and logic such as clock and accelerometer functions to provide input/triggers for sensing; and
9. Components configured to communicate to cloud or phone.

Because the jugular vein may be hard to find for a patient, a wearable ultrasound patch of the present disclosure may contain an array of sensors, such as ultrasound and/or other sensing element types including photoplethysmography (PPG) via light, so that the placement need not be as precise in order to obtain measurements. The received signals from the array of sensors can be analyzed to select the specific transducers to use for monitoring changes in vein dimensions with time.

Aspects of the present disclosure include a wearable patch designed to provide an acoustic impedance match between tissue and sensor array which may be achieved with the use of a gel patch adhered to the skin. A housing allows an electronics unit to be attached and removed while maintaining acoustic impedance match and position over the vein and consistency of scanning location. In another example a sensor may include a fluid-filled membrane. In another example the sensor is subcutaneously implanted to maintain contact with the tissue.

The sensor may contain a battery and a wireless communication module for wireless communication, e.g., Bluetooth or other, to phone or other edge computing device. Near Field Communication (NFC) or other wireless charging technique may be used to charge the sensor battery, take readings and communicate results. Alternative embodiments could use other technologies such as Radio Frequency (RF), Impedance, inductance, light, pressure sensors, etc.

The wearable ultrasound patch may be used in combination with a manoeuvre to distinguish between the jugular vein and an artery (a vein expands in size with breath hold while an artery does not). After identifying the vein, the geometry (area or diameter) of the vein may be extracted. In an example the data may be plotted over time, for example, a 60 second trace. The plot or data analysis may be used to estimate haemodynamic volume status. The sensor data may be monitored periodically, e.g., daily for trends or threshold crossing and the results can be communicated to the patient and to modify a treatment (prescription).

External ultrasound is technically difficult, expensive and requires large hardware. Recent developments have reduced the size and complexity of these systems, however they still require use by a trained operator to position the transducer accurately and interpret the output in order to measure a structure. They are also difficult to use for repeated daily measurements as the monitoring location is likely to vary. By contrast, sensors of the present disclosure are small, easy to use, initially applied by the physician but could also be applied by the patient either with training or use of marker tattoo or more permanent placement patch and also improve reproducibility as they are worn continuously for an extended period of time.

Episodic standard ultrasound sensors of the present disclosure provide continuous/regular intermittent monitoring. These sensors can be body worn for a period, e.g., 30 days, during which it cycles through sleep and monitoring modes at regular intervals which can be time-based or patient position/activity based. Valsalva or other manoeuvres can be performed and the sensor can contain audio cues to assist in the performance of the manoeuvre.

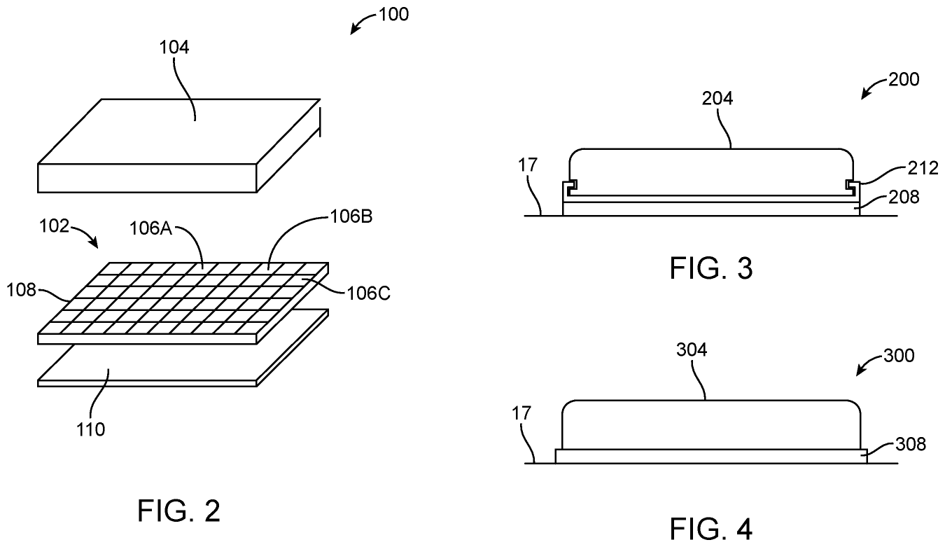

With reference to FIG. 2, an example of sensor 100 is shown which is configured for securement to the neck over the jugular vein. In the illustrated example, sensor 100 includes a transducer module 108 that includes a two-dimensional array 102 of independently controllable ultrasound transducer elements 106 (e.g., 106A-106C) for producing ultrasonic pulses. Transducer module 108 is capable of 2D and 3D B-mode scanning in addition to functional Doppler modes such as Pulsed Wave, Colour and Power Doppler. The array 102 of ultrasound transducer elements 106 enables imaging of the jugular vein, and assessment of both the anatomical structure and blood volumetric flowrates of the arterial and venous vessels, which can be measured anywhere, including in a home of the patient, to allow for long-term monitoring by a clinical team.

In the embodiment shown in FIG. 2, transducer module 108 comprises an array of N x M independently controlled ultrasonic elements 106, for example in piezoelectric crystal or capacitive micromachined ultrasonic transducers (CMUT) form. In some examples the elements 106 are capable of beam steering.

The ultrasound transducer module 108 in this embodiment has a length of 20 mm-100 mm and a width of 5 mm-50 mm with a plurality of piezo crystals/CMUT cells. Depending on patient size, the dimensions of the ultrasound transducer and the number of cells could vary. In some examples, sensor 100 includes a one-dimensional array of transducer elements 106 with a length configured and dimensioned to detect opposing walls of the jugular vein and, in some examples, adjacent structures. The one dimensional array of elements 106 are positioned along a transverse axis that is configured to be positioned generally perpendicular to a longitudinal axis of the jugular vein. In some examples, the one dimensional array may have a plurality of transducer elements, for example, four or more transducer elements. As shown in FIG. 2, additional rows of elements may be included in parallel to a first row of elements providing a two-dimensional array. Although FIG. 2 conceptually shows six rows of ten elements 106 positioned along corresponding transverse axes, this is for convenience of illustration only and any number of rows with any number of elements in each row may be used.

In an example the transmission frequency range of the ultrasound transducer varies between 7 MHz and 20 MHz, which may be tuned in an initial training phase, dependent on the size and shape of the patient. In other examples any frequency range known in the art of ultrasound sensing may be used.

In some examples the patient would periodically affix the sensor 100 to the body throughout a monitoring period. The transducer module 108 in this embodiment is secured to the neck by a pad 110 formed from an adhesive/gel/sponge-like substance. The pad 110 provides an ultrasound/acoustic transfer medium between the transducers and the skin.

Sensors of the present disclosure may include a mechanical housing with adhesive and an acoustic layer that are disposable and a reusable hardware system that can be attached and detached. The attachment could have a number of elements, for example, a plastic housing containing mechanical attachment and locking elements to connect to reusable hardware system. The sensor can also include an aperture through which a gel pad protrudes and contacts the array of ultrasound transducers when the reusable hardware module is engaged and attached to the disposable layer. The system may include an adhesive surface to attach a gel pad and be configured according to an acoustic impedance matching material method. The sensor includes a surface coupling material such as a gel. In an example, an adhesive gel layer is provided which attaches to the housing. The adhesive gel layer provides contact to the skin with adhesive layer and ensures compression of the gel layer when the reusable hardware system is attached. In some embodiments this mechanical housing may be curved in one plane to fit the natural contours of the neck.

FIG. 3 shows one embodiment of a sensor 200 with a pad 208 that allows for daily reuse of the ultrasound transducer. By reusable, it is meant that the pad 208 can remain on the skin for the entire monitoring period. The pad 208 is provided with a mechanical locking plate 212 adapted to removably receive ultrasound transducer module 204. In this embodiment the ultrasound transducer module 204 slides or clicks or clasps within the locking plate 212 to secure it to the underlying adhesive pad 208 and hence the neck, ready for use. Between readings, the ultrasound transducer module 204 can be removed from the patient by sliding it out from the locking plate 212 for recharging. The adhesive pad 208 incorporating the locking plate 212 may remain on the skin 17 until the next use without causing discomfort to the patient. Any of a variety of mechanical engagement features for removably attaching transducer module 204 to locking plate 212 may be used in addition to or instead of a sliding engagement, such as a pivoting engagement and one or more latch components of interlocking engagement.

As shown in FIG. 4, as an alternative embodiment, a sensor 300 includes a disposable gel or adhesive pad 308, which is intended to be disposed of after each use. The pad 308 has no locking plate. Instead, both the upper and lower surface of the pad 308 are adhesive to attach on one side to the transducer module 304 and on the other to the skin 17. The pad 308 is intended for one-time use and is completely removed from the skin when the transducer is not in use. In some examples, to ensure that the transducer is periodically replaced in the same location on the neck throughout the monitoring period, the adhesive pad 308 is provided with a plurality of guide holes, for example on the corners of the pad for alignment with corresponding markers on the skin by positioning the markers within the guide holes.

In some examples, sensor 300 is not designed to be adhered to the skin and other forms of securement of the transducer to the neck may be used such as belts or collars.

As shown in FIG. 2, sensor 100 further comprises an electronics module 104 (sensor 200 includes electronics module 204 and sensor 300 includes electronics module 304). As described above in connection with FIG. 1A, sensor 100 may further comprise a user interface (not shown) physically located on the sensor or in wireless communication with the sensor in order to control and communicate with the sensor, such as to start and stop/pause the examination procedure. The user interface may take the form of a button, a touch pad or other activation means. A signal generator (audio, visual or haptic buzzing etc.) may further be provided to prompt the patient to perform specific breathing maneuvers to enhance the clinical signal, for example natural breathing (inhale/exhale), sudden sniff or Valsalva. An accelerometer may also be provided to enable the system to track the motion and position of the patient.

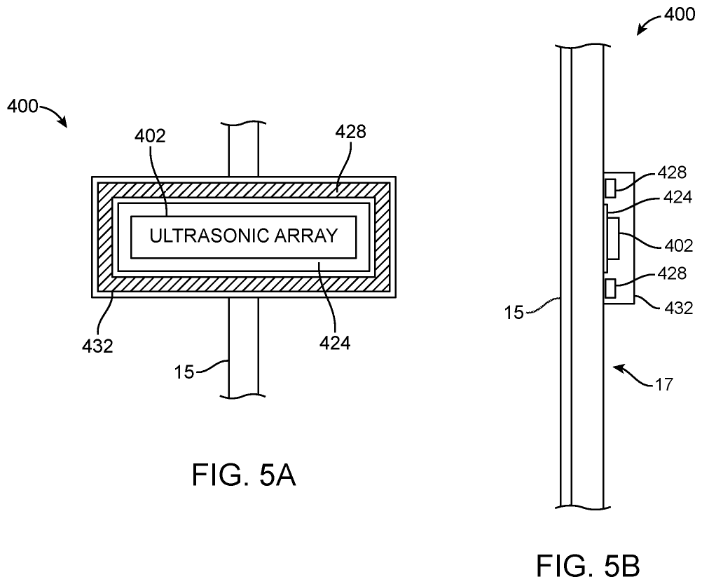

FIGS. 5A and 5B show another example of a sensor for use as a wearable ultrasound jugular vein patch. FIG. 5A is a plan view of a sensor 400 showing a 1-D ultrasound array 402 disposed over an acoustic coupling gel volume 424 proximate a vein 15. The gel volume 424 is surrounded by a border of adhesive 428 for attaching to skin 17 (as shown in FIG. 5B). In some examples, the gel volume 424 is an encapsulated gel with adhesive patch configured for acoustic matching. In other examples, a liquid gel patch is pierced during application of the patch and electronics to the skin. In yet other examples, gel is applied to a region of the skin and a patch with a perimeter of adhesive is applied over the region where the gel has been applied. In such examples the sensor may or may not also have a gel layer. The gel may be any acoustic coupling material known in the art of ultrasound sensing.

In the illustrated examples of FIGS. 5A And 5B, the transducer array, gel, and adhesive are located in a common housing 432. In other examples the transducer array may be located in a separate electronics module removably coupled to the housing. The array of transducers 402 may be supported by a flexible or rigid printed circuit board and the housing 432 may similarly be flexible or rigid.

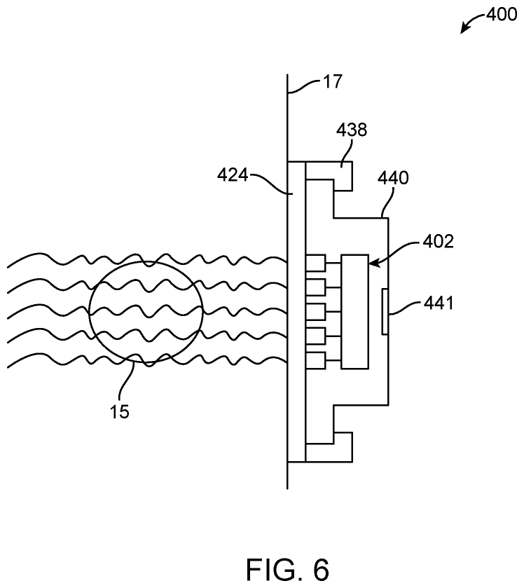

FIG. 6 illustrates another example implementation of a sensor 400 of the present disclosure that is adhered or otherwise attached to a surface of a patient's skin 17 adjacent the jugular vein 15 for sensing a dimension of the vein. As shown, the jugular vein is below the skin, a gel patch 424 is coupled to the skin 17, a patch housing 438 is adhered or otherwise coupled to the patch 424, an electronics housing 440 is attached to the patch housing 438 which contains electronics. An ultrasound transducer array 402 in the electronics housing 440 creates ultrasonic pulses (depicted as wavy lines). The electronics housing 440 also includes one or more user interface elements such as a button 441 and/or display screen for controlling the sensor 400.

Reflections occur in tissue and are detected by the transducers and the reflections are used to determine vein geometry. A button 441 or similar may be included for activating/controlling sensor 400.

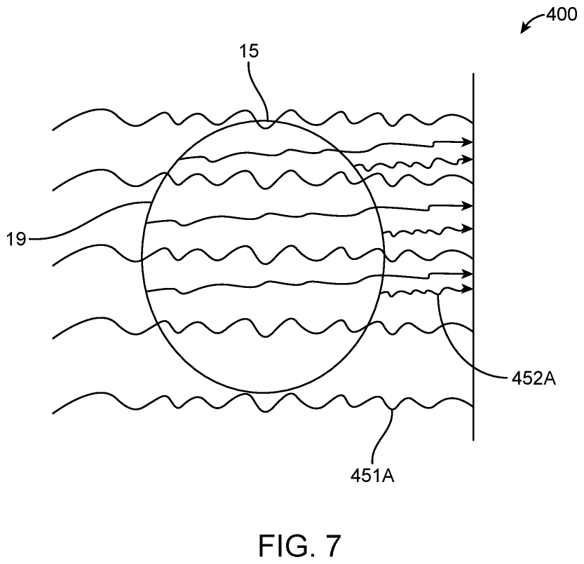

FIG. 7 conceptually illustrates an example of reflections from the vein walls 19 with the transducer waves 451 (e.g., 451A) and reflected waves 452 (e.g., 452A) from density changes in tissue and at vein walls. The reflected waves 452 are detected at sensor 400. The transmitted ultrasound wave travels at a wave speed characteristic for the tissue/fluid it travels through. At transition points from one type of tissue to another type of tissue, wave reflections occur. Those reflected waves are backpropagated to the receiver and arrive as an ‘echo’ at a certain echo time at the receiver. From the time of travel and an assumed average wave speed it is possible to allocate a geometric distance to the echo-in order to geometrically locate the reflection point (e.g. at the edge of the jugular vein where tissue changes from vascular tissue to blood). Practically two echoes would be required to derive the jugular vein diameter from those signals and certainty must exist that those echoes indeed stem from the vessel wall of the jugular vein and not from other tissue transitions.

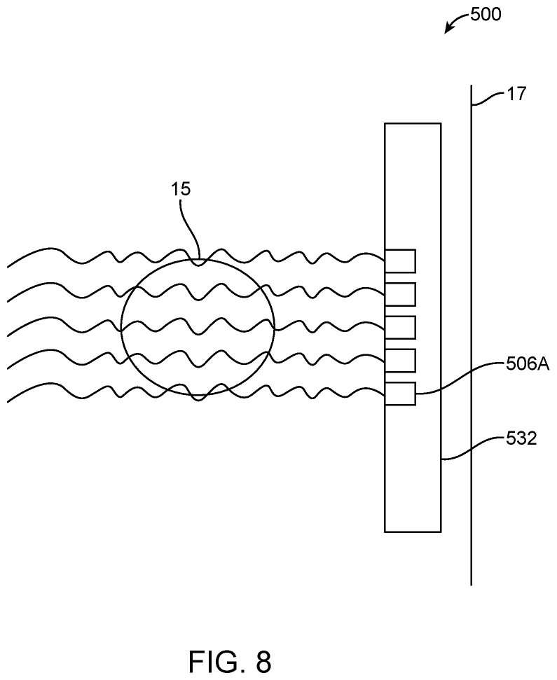

FIG. 8 illustrates another example of a wireless ultrasound sensor 500 made in accordance with the present disclosure. In the example shown in FIG. 8, the sensor 500 is a subcutaneous sensor implanted below a patient's skin 17 proximate the jugular vein 15. In this example sensor 500 does not have an acoustic matching layer such as the gel pad and also does not have two housing components that are removably attached. The sensor 500 shown in FIG. 8 has a housing 532 that contains an array of ultrasound transducers 506 (e.g., 506A) and associated electronics for controlling the transducers and receiving the reflected ultrasound signals as well as a battery and wireless communication module for communicating with an external computing device.

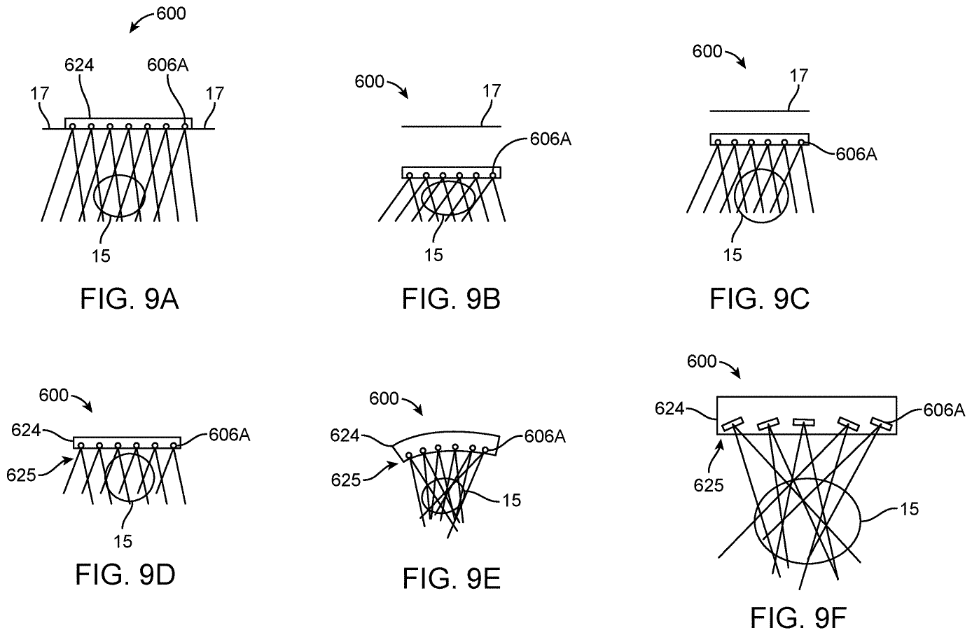

FIGS. 9A-9F show additional examples of sensors and the arrangement of the sensors and arrays of ultrasound transducers with respect to the targeted vein. FIGS. 9A-9C illustrate sensors 600 configured to be installed on a patient's skin 17 (FIG. 9A) or below a patient's skin 17 (9B, 9C) and may be configured to be disposed at various depths below the skin 17, including positioned on the jugular vein 15 in direct contact with the vein 15 (9B). Outside of the array of ultrasound transducers and core electronics needed to drive the transducers and receive the reflected signals, the surrounding housing and impedance-matching layers, and anchoring features for maintaining the sensor in a fixed position may vary between the examples shown in FIGS. 9A, 9B, and 9C. Some of these embodiments may contain lateral fixation features or protuberances to prevent rotation in the subcutaneous space. The curved housing 9E may require less significant features due to its curved shape resisting rotational motion in situ.

FIGS. 9D and 9E show two examples of transducer housings that may be utilized. In the example shown in FIG. 9D, the sensing side 625 of the housing 624 from which ultrasonic signals are transmitted and received is substantially flat or planar and the active side of each transducer element 606 (e.g., 606a) is parallel to the flat sensing side 625 of the housing 624. This arrangement will ensure that when positioned correctly over the target vein, some of the transducer elements will align with the vessel. Dimensions of the device 600 will be such that the beam profile of at least three elements 606 will cross a smallest portion of the monitored jugular vein, thus enabling the estimation and monitoring of the diameter and/or area of the vein. In FIG. 9E, the sensing side 625 of the housing 624 is curved and the active side of each transducer element 606 (e.g., 606a) is again substantially parallel to the housing resulting in a curved array of transducers. FIG. 9F has a similar resulting configuration of transducer elements as FIG. 9E, except the elements 606 are positioned at slightly varied acute angles to the sensing side 625 of the housing 624 to provide a plurality of angled positions for the sensing elements 606. These curved transducer arrangements allow for the beams of more transducers to cross the target vessel without having to pack them closer together. More measurements facilitate a more accurate estimation of diameter or area. In some examples the transducer arrays may be two dimensional and the relative positions of the transducer elements can be varied along a given transverse row of elements as well as across a given column of transducer elements. Electronics and software/firmware for controlling the transducer elements may be configured according to the physical arrangement of the transducer elements.

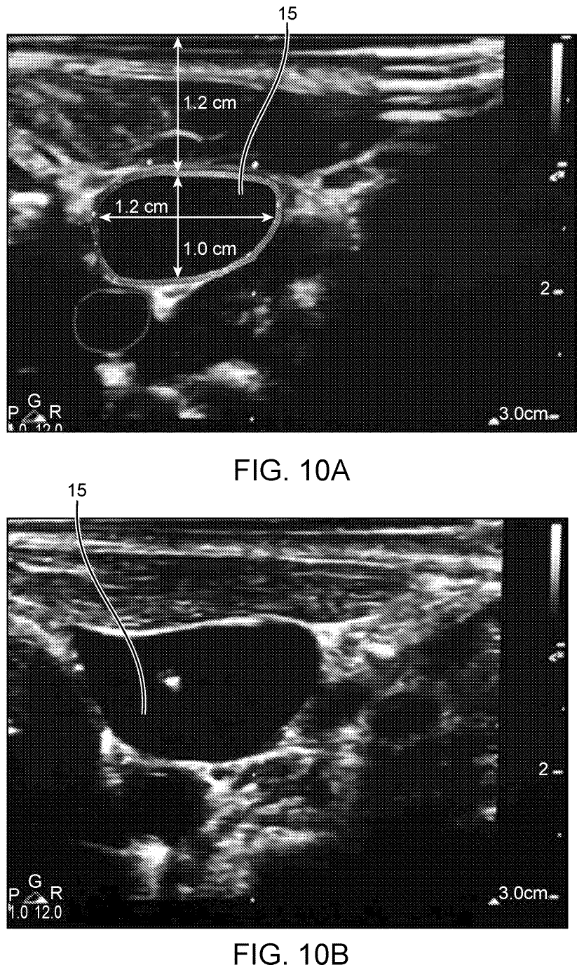

FIGS. 10A and 10B are example illustrative images from an ultrasound scan showing internal jugular vein 15 located 1-2 cm below a patient's skin (FIG. 10B is an enlarged version of FIG. 10A).

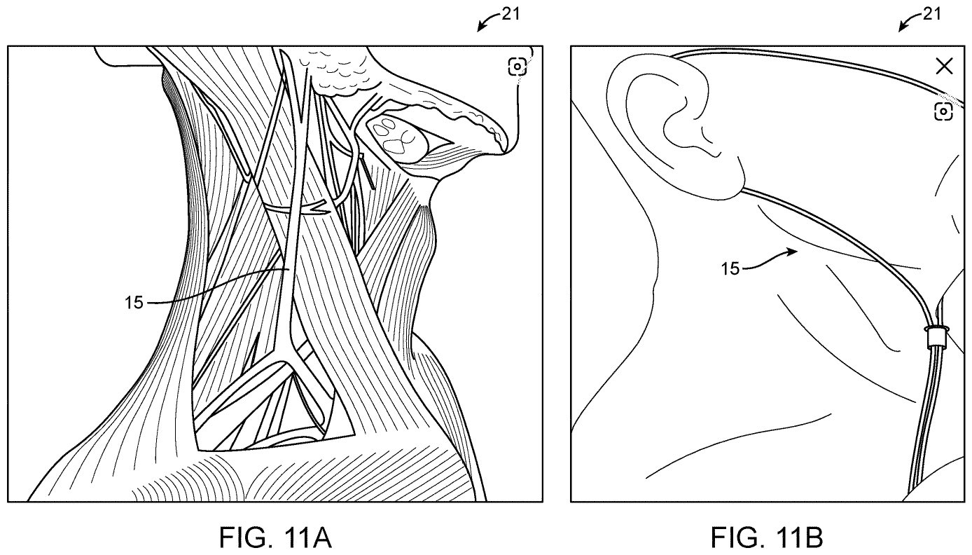

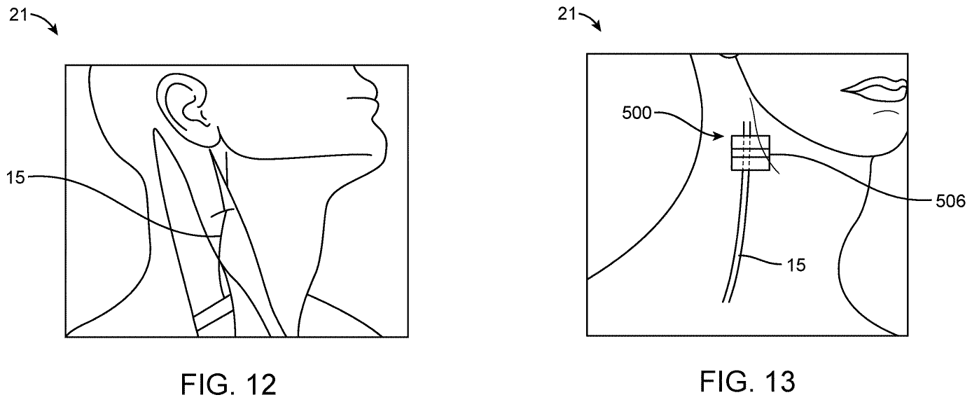

FIGS. 11A-12 show where the jugular vein 15 is located on a patient 21. FIG. 11B shows a hypervolemic patient's enlarged jugular vein 15 and thus the appropriate position an ultrasound device is located for monitoring a vessel. FIG. 13 illustrates an example placement of sensor 500 over the jugular vein 15 in a patient 21 for monitoring a diameter or other physical dimension of the vein over time. As shown in FIG. 13, the transducer element array 506 extends along a transverse axis and is positioned over the vein 15 such that the transverse axis of device 500 is approximately orthogonal to a central longitudinal axis of the vein 15.

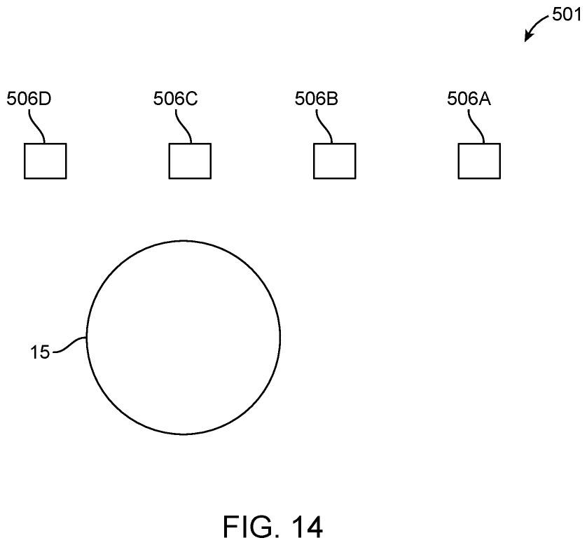

FIG. 14 conceptually illustrates a possible offset placement of the transducer array 501 of transducers 506 (e.g., 506A-506D) relative to the jugular vein 15 where a central vertical axis of the array 501 is offset from a central longitudinal axis of the vein 15. As will be appreciated, in a real world application such an offset arrangement may occur because the precise location of a patient's jugular vein can be challenging to determine and because sensors of the present disclosure may be used by untrained individuals such as the patient. The sensor and associated electronics may be configured to identify the jugular vein despite the offset location to determine a baseline vein dimension and for ongoing monitoring of the vein.

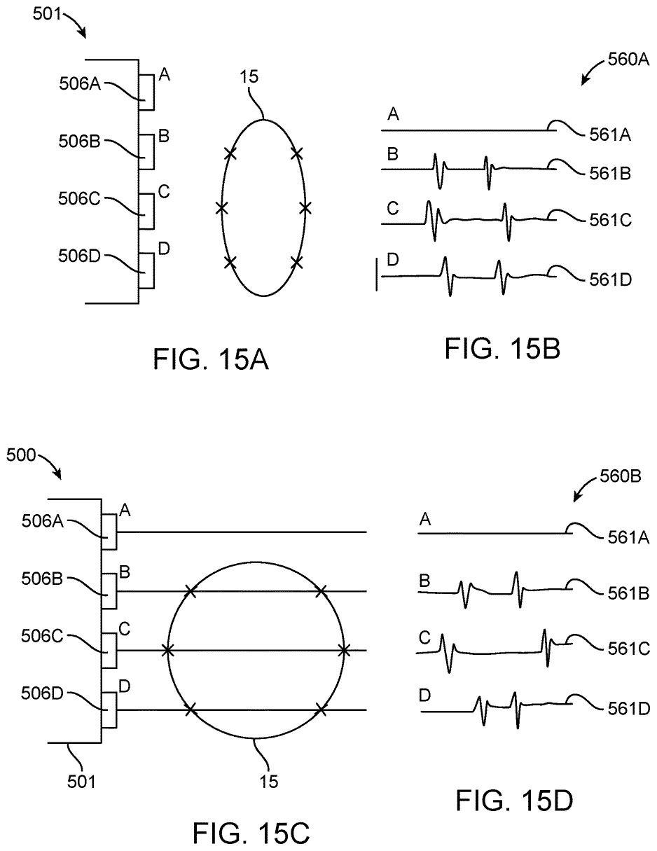

FIGS. 15A-15D conceptually illustrate an ultrasound transducer array 501 of a sensor 500 proximate to vein 15 with resultant signal recordings. Array 501 includes a plurality of transducers 506 (506A-506D) as shown in FIGS. 15A and 15C. A conceptual illustration of a temporal signal 560 (560A and 560B), in the form of waveform ultrasound recordings, received from sensor 500 is illustrated in FIGS. 15B and 15D. The plurality of transducers 506 produce corresponding signals 561A-561D, which include peaks that indicate density changes encountered by the ultrasonic waves and may correspond with locations where the ultrasonic waves interacted with vessel walls (those locations are represented by “X”s in FIGS. 15A and 15C). The temporal signal 561 may be displayed on a display screen of an external computing device in wireless communication with the sensor for recording of the reflected waves at the transducer elements. A compressed/collapsed vein is shown in FIG. 15A and an inflated or expanded vein in FIG. 15C. In the illustrated example the signal 561C from transducer 506C has the largest time duration between the voltage peaks, indicating the largest distance between the opposite sides of the vessel wall, which distance is larger when the jugular vein is expanded as can be seen from FIG. 15D. The signal from transducer 506C can be used to determine a dimension of the vein 15 in a first direction parallel to the propagation direction of the transducer pulse. The vessel shape can be inferred by matching the intersections of all of the beams, 506A through 506D. The shape may be circular or oval depending on the state of the vein collapse or other physiological factors.

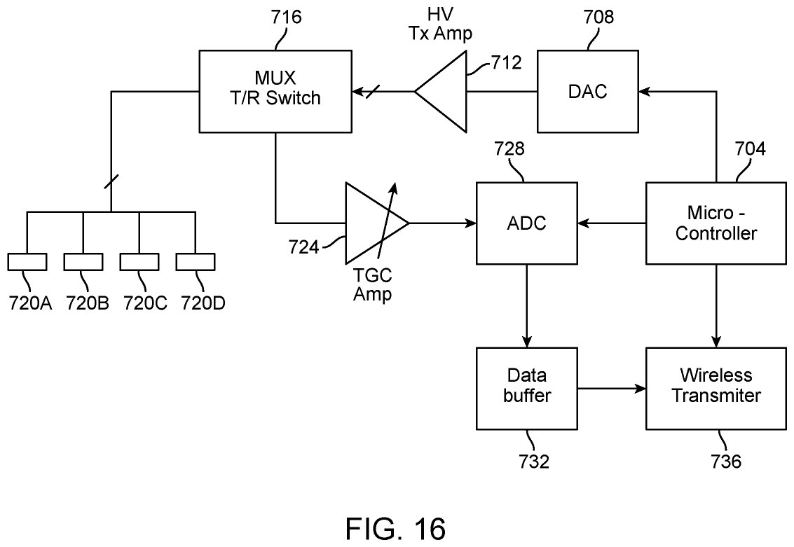

FIG. 16 is a functional block diagram of components of a sensor according the present disclosure. In the illustrated example, to transmit ultrasonic pulses, a microcontroller 704 sends out a pulse which is converted by a digital analog converter (DAC) 708. The signal is fed into a high voltage transmit amplifier (HV Tx Amp) 712. A multiplexer (MUX) element 716 switches on each transducer 720 (e.g., 720A-720D) using a transmit/receive switch. The illustrated example includes a one-dimensional array of four ultrasound transducer elements 720, each may contain any of a variety of standard ultrasound element constructions known in the art. In examples of sensors that are designed to be implanted, due to the implanted nature of the system, there is no need for an impedance-matching material such as a gel. The electrical signal is converted to mechanical energy at specific signal and this mechanical (ultrasound) wave is emitted.

To receive reflected ultrasonic signals, the transducer elements 720 that had transmitted the ultrasonic pulses are then excited by the reflected ultrasonic pressure waves and the received mechanical energy from the reflected waves is converted to electrical signals by the transducer elements 720. The multiplexer 716 switches to a receive chain for receiving and transmitting the reflected signal. The received signal is transmitted to a time gain compensation amplifier (TGC amp) 724 which increases the gain proportional to the depth as the signal is detected to amplify the received signal. The amplified analog signal is converted to a digital signal by an analog digital converter (ADC) 728. The digital signal is fed into a data buffer 732 and transferred to the wireless transmitter 736 module for communication to an external processing unit/reader.

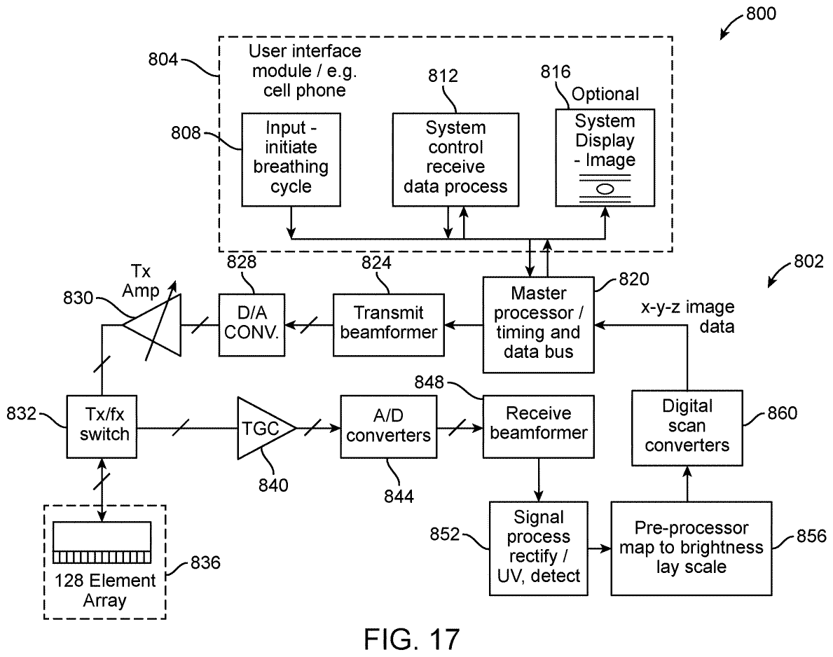

FIG. 17 is a functional block diagram of a system 800 for implementing aspects of the present disclosure. System 800 includes a user electronic device 804 in communication with sensor 802, which may be attached to or implanted subcutaneously in a patient proximate a vein. Electronic device 804 receives input from the user and displays information received from the sensor 802, such as images of the monitored vessel, measured vessel parameters, warnings or alerts, and recommendations to begin, end, or modify various treatments. Device 804 may include a user input module 808 that allows a user to initiate a breathing cycle or similar, a system control 812 for communicating with sensor 802 and processing data received from sensor 802 and/or user input or data from other sources. An optional display 816 can display images of the vessel or other related information.

Sensor 802 includes a master processor/data bus 820, a beamformer 824, a digital analog converter 828, a high voltage transmit amplifier (HV Tx Amp) 830, a transmit/receive switch 832 connected to an array 836 of ultrasound transducers as well as a time gain compensation amplifier (TGC amp) 840, which increases the gain proportional to the depth as the signal is detected to amplify the received signal. The amplified analog signal is converted to a digital signal by an analog digital converter (ADC) 844. The digital signal is fed into a beamformer 848 and transferred to a signal processor 852 then to pre-processor 856 before a digital scan converter 860, which generates image data. The image data can be sent to the master processor, which can relay the images to electronic device 804.

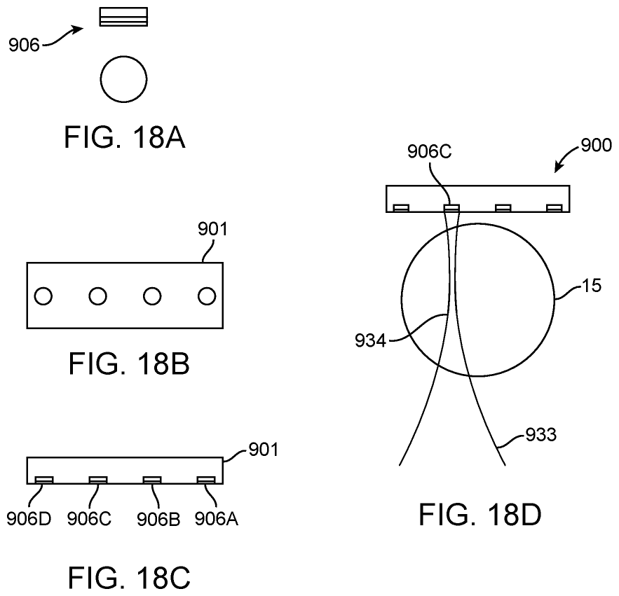

FIGS. 18A-18D illustrate one example of a device 900 containing a one dimensional array 901 of transducer elements 906 and the array's position with respect to a monitored vessel 15. The device is sealed and also contains the electronics to run the system, the battery and the communication elements to communicate to the external receiver part of the system (e.g., as discussed above with respect to FIGS. 16-17). In the illustrated example the array 901 includes four transducer elements 906 (906A-906D). FIG. 18A shows a single element transducer 906. FIGS. 18B and 18C show a front and side view of a four transducer array 901. FIG. 18D schematically shows device 900 centered over a vein 15 and a beam profile 933 from transducer 906C with respect to the vein 15.

FIG. 18D shows the spacing of transducer elements 906 and depicts the total length of the array 901 relative to a jugular vein 15. The length of the array 901 is greater than a diameter of the jugular vein 15 for ensuring the detection of the outer walls of the vein. FIG. 18D also shows that the beam profile 933 of each transducer element 906 contracts to a narrowed portion 934 before expanding. In the illustrated example the array 901 is spaced from the jugular vein 15 so that the narrowed portion of the beam profile 933 is at a depth below the skin surface that is approximately the same as a central longitudinal axis of the vein 15.

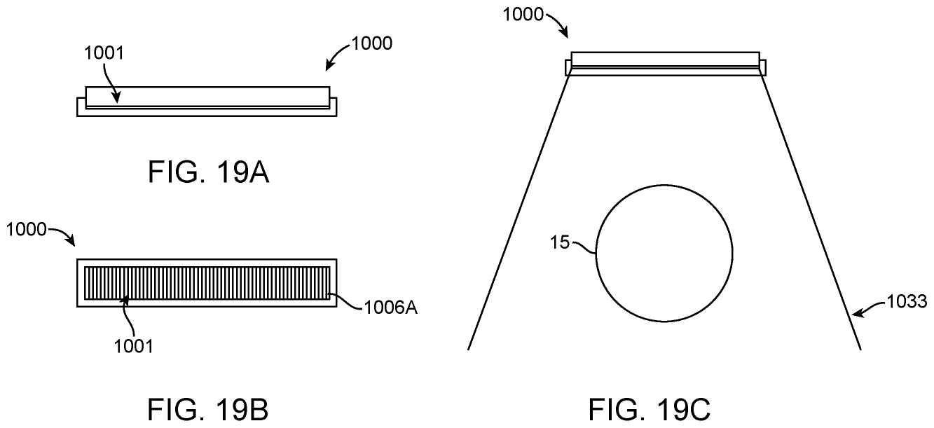

FIGS. 19A-19C illustrate another example of a package 1000 containing a one-dimensional array 1001 of transducer elements 1006 (1006A) and the array's position with respect to a monitored vessel 15. In the illustrated example the array 1001 includes 128 transducer elements 1006. FIG. 19C shows the spacing of the transducer elements 1006 and total length of the array 1001 relative to a jugular vein 15. The length of the array 1001 is greater than a diameter of the jugular vein 15 for ensuring the detection of the outer walls of the vein 15. FIG. 19C also shows the aggregate image plane 1033 of the array 1001. FIG. 19C also shows the aggregate image plane of the array which widens with distance from the array 1001 and covers an area that includes an entirety of the vein. With a full electronic array, the image is formed of many individual scan lines with the ability to steer and focus the beams with high fidelity. Thus, the vessel wall can be well defined, leading to an accurate assessment of the vessel shape and size.

An ultrasound transducer such as that described above may be positioned and secured on the patient. To enable monitoring, the ultrasound transducer is placed on the same site of the patient daily or for a period of days. This could be assisted with the use of tattooing of the skin for repeatable placement of the transducer. Specific placement of the transducer could be decided upon on a patient-by-patient basis, depending on their anatomical profile and determined during an initial training/educational phase (for example in the hospital in consultation with healthcare professional). For remote monitoring, measurements are taken at the same time each day, with the patient in the same position, to ensure repeatability.

A device may be provided that auto locates the same jugular vein segment over time. A mechanical or similar set-up that ensures that the device is over same approximate area (for example, this might be a belt with a dedicated holder and straps to ensure repeatable positioning).

Alternatively, or additionally, inked (permanent or the likes of semi-permanent) markers on the patient's body could be used for positioning. The device could recognize it is in the correct location using optical recognition of, for example, three tattooed dots on the patient's body. This may be assisted by the device comprising a handheld probe shaped like a computer mouse.

A plurality of tattooed dots (or other shapes—such as asymmetric triangles such that both location and orientation can be precisely tracked)—either permanently or temporary may be used. These may be stenciled on in a doctor's office or be applied via ink on an adhesive positioned by a trained operator. By providing such alignment features on the patient's neck, consistent orientation and location of the sensor can be achieved over a number of measurements, which can help ensure consistent measurements of the same location of the jugular vein. In use, the holder for that day's reading may be lined up with those dots and then affixed using some type of adhesive.

Any holder set-up straps or similar could be released and the individual let breathe freely during the reading.

The above may be coupled with feature extraction (e.g., carotid artery or other anatomical recognition) to identify the precise cross-sectional area (CSA) required. Such a device would be able to locate the same jugular vein CSA consistently over extended periods of monitoring—months to years—and capture during relative motion of respiration cycles (combination of device itself moving with respiration and image analysis). Such a device could trigger a series of prompts for a patient to engage in appropriate manoeuvres (either a set sequence and/or selected using some decision-making criteria). For example, readings on a first day may include 25 mm maximum diameter for the jugular vein and a collapse of 5 mm. Readings on the next day may include 24 mm maximum diameter for the jugular vein and a collapse of 2 mm. A processor may be configured to execute the steps of an algorithm that detects the reduced collapse at the same area (which could be a sign of volume overload but could be indicative of a hypovolemic state (depending on what area is ‘normal’)).

The steps of the algorithm may include identifying a manoeuvre to test the cause of the reduced collapse, such as a sniff test by instructing the patient to take a reading with a sniff in the middle to determine the optimal sequence to personalise the person on his/her own P-V curve—continuously gather/acquire images during the manoeuvre/activity, which may be useful for HFpEF patients in particular; and quasi stress-test (e.g., insight into splanchnic capacitance).

The steps of the algorithm may also include generating a jugular vein score which may be used to provide therapeutic input. An extended duration patch may be provided for continuous monitoring, which may have a rechargeable battery and/or may fit into a holder.

Any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

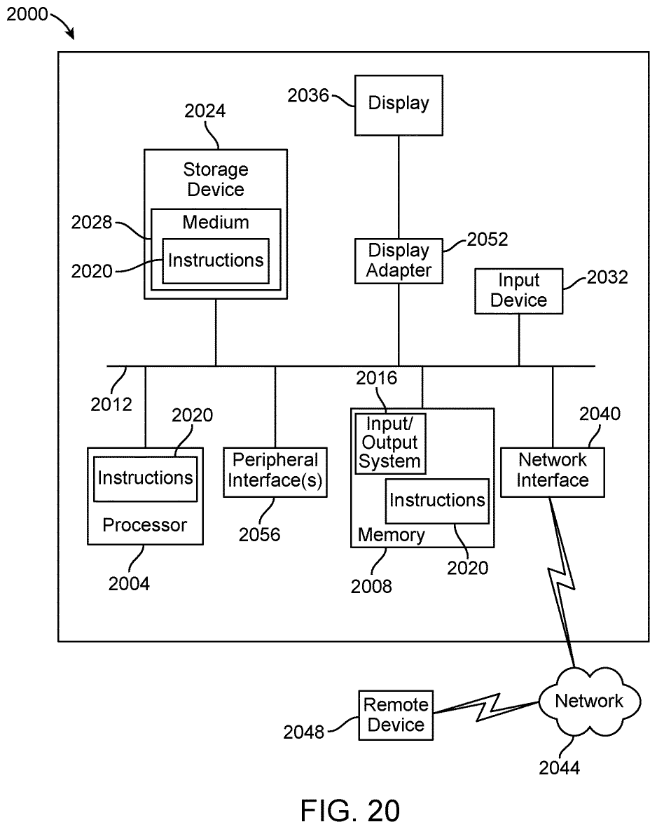

FIG. 20 shows a diagrammatic representation of one embodiment of a computing device in the exemplary form of a jugular vein diameter/area measuring implant control and communication system 2000 within which a set of instructions for causing an implant control and communication system, such as a waveform generator, an oscilloscope, an EFM circuit, or an implant, among other systems and devices disclosed herein, to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 2000 includes a processor 2004 and a memory 2008 that communicate with each other, and with other components, via a bus 2012. Bus 2012 may include any of several types of bus structures including, but not limited to, a memory bus, a memory controller, a peripheral bus, a local bus, and any combinations thereof, using any of a variety of bus architectures.

Memory 2008 may include various components (e.g., machine-readable media) including, but not limited to, a random access memory component, a read only component, and any combinations thereof. In one example, a basic input/output system 2016 (BIOS), including basic routines that help to transfer information between elements within computer system 2000, such as during start-up, may be stored in memory 2008. Memory 2008 may also include (e.g., stored on one or more machine-readable media) instructions (e.g., software) 2020 embodying any one or more of the aspects and/or methodologies of the present disclosure. In another example, memory 2008 may further include any number of program modules including, but not limited to, an operating system, one or more application programs, other program modules, program data, and any combinations thereof.

Computer system 2000 may also include a storage device 2024. Examples of a storage device (e.g., storage device 2024) include, but are not limited to, a hard disk drive, a magnetic disk drive, an optical disc drive in combination with an optical medium, a solid-state memory device, and any combinations thereof. Storage device 2024 may be connected to bus 2012 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 2024 (or one or more components thereof) may be removably interfaced with computer system 2000 (e.g., via an external port connector (not shown)). Particularly, storage device 2024 and an associated machine-readable medium 2028 may provide nonvolatile and/or volatile storage of machine-readable instructions, data structures, program modules, and/or other data for computer system 2000. In one example, software 2020 may reside, completely or partially, within machine-readable medium 2028. In another example, software 2020 may reside, completely or partially, within processor 2004.

Computer system 2000 may also include an input device 2032. In one example, a user of computer system 2000 may enter commands and/or other information into computer system 2000 via input device 2032. Examples of an input device 2032 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 2032 may be interfaced to bus 2012 via any of a variety of interfaces (not shown) including, but not limited to, a serial interface, a parallel interface, a game port, a USB interface, a FIREWIRE interface, a direct interface to bus 2012, and any combinations thereof. Input device 2032 may include a touch screen interface that may be a part of or separate from display 2036, discussed further below. Input device 2032 may be utilized as a user selection device for selecting one or more graphical representations in a graphical interface as described above.

A user may also input commands and/or other information to computer system 2000 via storage device 2024 (e.g., a removable disk drive, a flash drive, etc.) and/or network interface device 2040. A network interface device, such as network interface device 2040, may be utilized for connecting computer system 2000 to one or more of a variety of networks, such as network 2044, and one or more remote devices 2048 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 2044, may employ a wired and/or a wireless mode of communication. In general, any network topology may be used. Information (e.g., data, software 2020, etc.) may be communicated to and/or from computer system 2000 via network interface device 2040.

Computer system 2000 may further include a video display adapter 2052 for communicating a displayable image to a display device, such as display device 2036. Examples of a display device include, but are not limited to, a liquid crystal display (LCD), a cathode ray tube (CRT), a plasma display, a light emitting diode (LED) display, and any combinations thereof. Display adapter 2052 and display device 2036 may be utilized in combination with processor 2004 to provide graphical representations of aspects of the present disclosure. In addition to a display device, computer system 2000 may include one or more other peripheral output devices including, but not limited to, an audio speaker, a printer, and any combinations thereof. Such peripheral output devices may be connected to bus 2012 via a peripheral interface 2056. Examples of a peripheral interface include, but are not limited to, a serial port, a USB connection, a FIREWIRE connection, a parallel connection, and any combinations thereof.

Embodiments described herein may provide a number of beneficial effects and advantages, including the following. The devices and methods described may function as a fluid status indicator. Patients can be managed with greater confidence in euvolemia—i.e. with a greater margin of safety. The physician can take enough fluid off to restore some venous capacitance to act as a buffer against sudden fluid overload causing an acute decompensation—without taking so much fluid off as to cause kidney issues because jugular vein Volume Metric and collapsibility (jugular vein CI) are both key measures of patient's fluid status, and are more sensitive/responsive than pressure.

The devices and methods described may function as a decompensation risk indicator. Since jugular vein diameter or area measurements (e.g. diameter) increase/jugular vein collapsibility decreases (relative to an individual patient's baseline), such measurements can provide an earlier indicator of worsening fluid status, which in turn drives hemodynamic congestion, which drives clinical congestion (which may result in ADHF).

The devices and methods described may function as an aid to therapeutic decision making. Healthcare providers can use jugular vein Volume Metrics to determine optimal diuresis point with an ability to provide longitudinal measures over a period of hours/days/weeks, helping the physician to factor in the impact of fluid redistribution (e.g., from the interstitial tissue into the intravascular space).

The devices and methods described may function as another aid to therapeutic decision making because jugular vein Volume Metrics can assist healthcare providers in decision making as to whether to alter relative dosages of diuretics vis-à-vis vasodilators. For example, when a patient's cardiac pressure is increased, disclosed systems and methods facilitate important clinical decisions such as whether the cause is increased volume or increased vasoconstriction, whether to increase diuretics or vasodilators, or whether to use jugular vein Volume Metrics to rule in/out increased volume as a primary cause of increased pressures, i.e., if increased volume is confirmed then diuretics may be indicated, if not then vasodilators may be indicated.

Systems and methods disclosed may also:
assess jugular vein metrics;
compare daily results and trends to guideline based limits (and over time patient specific limits);
determine if medication modification is required;
send a signal/message to patient and requests confirmation of medication alteration;
determine exceptions (based on multiple times exceeding limits/trends/other trigger);
send notification to managing physician, who can then use system to send a message to patient to modify medication and confirm change;
provide alarms to remind patient to take medication/take reading; and/or
store all relevant information in the cloud server.

The foregoing has been a detailed description of illustrative embodiments of the disclosure. It is noted that conjunctive language such as is used herein in phrases like “at least one of X, Y and Z” and “one or more of X, Y, and Z,” unless specifically stated or indicated otherwise, shall be taken to mean that each item in the conjunctive list can be present in any number exclusive of every other item in the list or in any number in combination with any or all other item(s) in the conjunctive list, each of which may also be present in any number. Applying this general rule, the conjunctive phrases in the foregoing examples in which the conjunctive list consists of X, Y, and Z shall each encompass: one or more of X; one or more of Y; one or more of Z; one or more of X and one or more of Y; one or more of Y and one or more of Z; one or more of X and one or more of Z; and one or more of X, one or more of Y and one or more of Z.

Various modifications and additions can be made without departing from the spirit and scope of this disclosure. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present disclosure. Additionally, although particular methods herein may be illustrated and/or described as being performed in a specific order, the ordering is highly variable within ordinary skill to achieve aspects of the present disclosure. Accordingly, this description is meant to be taken only by way of example, and not to otherwise limit the scope of this disclosure.

Exemplary embodiments have been disclosed above and illustrated in the accompanying drawings. It will be understood by those skilled in the art that various changes, omissions and additions may be made to that which is specifically disclosed herein without departing from the spirit and scope of the present disclosure.

1-21. (canceled)

22. A system for monitoring a blood vessel of a patient, comprising:
a sensor including:
a housing;
a wireless transmitter;
an array of ultrasonic transducers attached to a first side of the housing;
a microcontroller in the housing connected to the array of ultrasonic transducers,
wherein the array of ultrasonic transducers is arranged such that, when the sensor is positioned proximate the blood vessel, an aggregate ultrasonic image plane that encompasses a cross section of the blood vessel is scannable by the array of ultrasonic transducers, and
a user electronic device in communication with the wireless transmitter, wherein the user electronic device includes a display with a user interface and is configured to receive data from the sensor.

23. The system of claim 22, wherein the microcontroller includes a module configured to process signals representative of ultrasonic waveforms and convert the signals to image data.

24. The system of claim 22, wherein the microcontroller includes a module configured to process signals representative of ultrasonic waveforms and convert the signals to a plurality of dimensional blood vessel parameters.

25. The system of claim 23, wherein the module is configured to process the image data to determine a plurality of dimensional blood vessel parameters, the plurality of dimensional blood vessel parameters including a blood vessel diameter and a blood vessel area.

26. The system of claim 24, wherein the module is configured to determine a change in one or more of the plurality of dimensional blood vessel parameters over time.

27. The system of claim 22, wherein the user electronic device is configured to display content based on data received from the sensor.

28. The system of claim 24, wherein the microcontroller includes an euvolemic module configured to compare a blood vessel diameter value to a range of diameter threshold values and compare a blood vessel area value to a range of threshold area values.

29. The system of claim 28, wherein the euvolemic module is configured to transmit a warning signal to the user electronic device when the blood vessel diameter value and/or the blood vessel area value are determined to be in ranges associated with a hypervolemic state or a hypovolemic state for the patient.

30. The system of claim 22, wherein the sensor is configured to be implanted subcutaneously.

31. The system of claim 30, wherein the array of ultrasonic transducers includes a plurality of transducer elements, wherein each of the plurality of transducer elements is attached to the first side of the housing, and wherein the first side of the housing is curved in one plane and, when the sensor is implanted proximate the blood vessel, the first side faces the blood vessel.

32. The system of claim 30, wherein the array of ultrasonic transducers includes a plurality of transducer elements, wherein each of the plurality of transducer elements is attached to the first side of the housing, and wherein one or more of the plurality of transducer elements is positioned at an acute angle with respect to a plane of the first side of the housing, and when the sensor is implanted proximate the blood vessel, the first side faces the blood vessel.

33. The system of claim 30, wherein the array of ultrasonic transducers includes a plurality of transducer elements, wherein each of the plurality of transducer elements is attached to the first side of the housing, wherein each of the plurality of transducer elements is configured to emit a beam profile having a narrowed portion, and wherein the narrowed portion is formed at a distance from the first side of the housing such that the narrowed portion is positioned near a central longitudinal axis of the blood vessel when the device is attached to or implanted in the patient.

34. The system of claim 22, wherein the sensor is configured to be attached to the skin of the patient.

35. The system of claim 22, wherein the array of transducers includes a two dimensional grid of a plurality of transducer elements.

36. The system of claim 34, wherein the housing includes a pad for attaching to skin, a locking plate attached to the pad, and an ultrasound transducer module containing the array of ultrasonic transducers, wherein the locking plate is configured to removably receive the ultrasound transducer module.

37. The system of claim 30, wherein the sensor includes a sensing electrode on the housing configured to detect sympathetic nervous signals.

38. The system of claim 30, wherein the sensor includes a pair of electrodes on the housing configured to detect tone by impedance between the pair of electrodes.

39. The system of claim 30, wherein the sensor includes one or more protuberances extending from the housing and sized and configured to reduce rotation within subcutaneous space.

40. The system of claim 22, wherein the housing contains a digital analog converter, a high voltage transmit amplifier, a multiplexer element, a transmit/receive switch, a time gain compensation amplifier, an analog digital converter, and a data buffer.

41. The system of claim 25, wherein the microcontroller includes a comparison module configured to compare the blood vessel area to a range of threshold area values for the patient and the blood vessel diameter to a range of threshold diameter values for the patient.

42. The system of claim 41, wherein the comparison module is configured to send instructions to send a warning to the user electronic device if the area is determined to be within a hypervolemic range of threshold area values, if the diameter is determined to be within a hypervolemic range of threshold diameter values, if the area is determined to be within a hypovolemic range of threshold area values, or if the diameter is determined to be within a hypovolemic range of threshold diameter values.

43. The system of claim 42, wherein the comparison module is configured to determine whether the patient is in a hypovolemic state, a euvolemic state, or a hypervolemic state based on the area and the diameter.

44. The system of claim 42, wherein the warning includes a prompt to initiate a treatment or medication change based on whether the patient is determined to be in a hypovolemic state, a euvolemic state, or a hypervolemic state.

45. The system of claim 44, wherein, when the patient is determined to be in the hypovolemic state, the treatment or medication change includes a recommendation for administration of fluids or vaso-constricting drugs.

46. The system of claim 44, wherein, when the patient is determined to be in the hypovolemic state, the treatment or medication change includes a recommendation for dialysis or administration or treatment guidance for diuretics or vasodilating drugs.

47-62. (canceled)
